(19) United States
(12) Patent Application Publication
(10) Pub. No.: US 2025/0146223 A1
(43) Pub. Date: May 8, 2025

(54) COMPOSITE MYCELIUM MATERIALS WITH ENHANCED MECHANICAL AND AESTHETIC PROPERTIES, AND EMBOSSING METHODS FOR PRODUCING SAME

(71) Applicant: BOLT THREADS, INC., Emeryville, CA (US)

(72) Inventor: Steven Tom, Emery, CA (US)

(21) Appl. No.: 18/730,006

(22) PCT Filed: Jan. 19, 2023
(86) PCT No.: PCT/US2023/060911

§ 371 Date: Jul. 18, 2024

Related U.S. Application Data
(60) Provisional application No. 63/301,334, filed on Jan. 20, 2022.

(51) Int. Cl. D21H 13/34; B31F 1/07; D21H 11/18; D21H 13/08; D21H 17/20; D21H 21/18; D21H 27/02

(52) U.S. Cl. CPC D21H 13/34; B31F 1/07; D21H 11/18; D21H 13/08; D21H 17/20; D21H 21/18; D21H 27/02

(57) ABSTRACT

Provided herein are mycelium materials and methods for production thereof. In some embodiments, a mycelium material includes one or more deformation areas spaced apart from each other. Additives and bonding agent may be combined with the mycelium material. Wet embossing methods of producing a mycelium material are also provided.

### CROSS REFERENCE TO RELATED APPLICATION

The benefit of priority to U.S. Provisional Patent Application No. 63/301,334 filed Jan. 20, 2022 is hereby claimed and the disclosure is incorporated herein by reference in its entirety.

### FIELD OF THE INVENTION

The present disclosure generally relates to various mycelium materials having a mycelium component and methods for production thereof to provide favorable mechanical and aesthetic qualities.

### BACKGROUND

Due to its bioefficiency, strength, and low environmental footprint, mycelium is of increasing interest in the next generation of sustainable materials. To this end, various applications have discussed methods of manufacturing mycelium materials. However, the mycelium materials currently undergoing development can have poor mechanical qualities, including susceptibility to delamination and tearing under stress, and non-uniform aesthetic qualities. What is needed, therefore, are improved mycelium materials with favorable mechanical properties, aesthetic properties, and other advantages, as well as materials and methods for making improved mycelium materials.

### BRIEF SUMMARY

It is an object of the invention to provide mycelium materials with enhanced mechanical and aesthetic properties, and embossing methods for the production thereof.

In accordance with an aspect of the invention there is provided a mycelium material comprising mycelium fibers, the mycelium material being planar and having a first surface, a second surface opposite the first surface, and a thickness. The first surface has one or more deformation areas spaced apart from each other, and the one or more deformation areas do not extend from the first surface to the second surface.

In accordance with another aspect of the invention there is provided a mycelium material comprising mycelium fibers, the mycelium material being planar and having a first surface, a second surface opposite the first surface, and a thickness. The mycelium material has a first region having a first density, and a second region having a deformation area extending from the first surface toward the second surface. The second region has a second density that is higher than the first density.

In accordance with an additional aspect of the invention there is provided a method of producing a mycelium material. The method comprises providing mycelium fibers; adding a solution to the mycelium fibers to produce a mycelium composition; wet embossing the mycelium composition such that the one or more deformation areas are formed; and drying the mycelium composition to produce the mycelium material.

The advantages and features of the present invention will become better understood with reference to the following more detailed description and claims taken in conjunction with the accompanying drawings.

### BRIEF DESCRIPTION OF THE DRAWINGS

FIG. 1A-1D illustrate a schematic overview of the wet emboss process in accordance with one embodiment of the invention.

FIGS. 2A and 2B illustrate scanning electron micrographs showing example cross sections of standard and wet emboss crust materials in accordance with one embodiment of the invention.

FIG. 3A-3B illustrate a schematic showing a wetlay over a mold placed on the forming cloth in accordance with one embodiment of the invention.

FIG. 4A-4B illustrate a schematic showing wetlay with a mold placed on top of the slurry in accordance with one embodiment of the invention.

FIG. 5A-5B illustrate a schematic showing wetlay with a mold extending through the thickness of the slurry/web in accordance with one embodiment of the invention.

FIG. 6A-6B illustrate the flexural moduli and drape of samples made with and without wet embossing in accordance with one embodiment of the invention.

FIG. 7A-7D illustrate scanning electron micrographs of standard and wet embossed samples subjected to manual peel in accordance with one embodiment of the invention.

FIG. 8A-8D illustrate representative force-travel plots from T-peel tests performed on standard and wet embossed samples in accordance with one embodiment of the invention.

FIG. 9A-9D illustrate force-travel plots for a wet emboss sample with unidirectional pleats in accordance with one embodiment of the invention.

FIG. 10A-10B illustrate a web wetlaid into a 3.2 mm thick mold with arrays of 3 mm, 6 mm, and 9 mm diameter holes in accordance with one embodiment of the invention.

FIG. 11 illustrates a heatmap of feature forming ability as a function of straight-walled hole diameter and depth in accordance with one embodiment of the invention.

FIG. 12A-12D illustrate the effect of drafted mold features on slurry infiltration in accordance with one embodiment of the invention.

FIG. 13A-13D illustrate a schematic showing effect of feature spacing on drape in accordance with one embodiment of the invention.

FIG. 14 illustrates the drape grades for samples embossed with 3D printed pillar array molds in accordance with one embodiment of the invention.

FIG. 15 illustrates dry tensile strength and elongation at break of samples made in different molds in accordance with one embodiment of the invention.

FIG. 16A-16C illustrate average T-peel force for wet embossed samples and schematics showing how the maximum degree of undulation may relate to sample thickness, in accordance with one embodiment of the invention.

FIG. 17A-17C illustrate scanning electron micrographs of a bridge bubble formed on a sample with the finish layer applied to the embossed side in accordance with one embodiment of the invention.

FIG. 18A-18D illustrate examples of finished wet emboss samples with the emboss pattern showing through the finish layer in accordance with one embodiment of the invention.

FIG. 19A-19F illustrate a schematic of how mold feature height affects surface smoothness in accordance with one embodiment of the invention.

FIG. 20A-20B illustrate examples of top surface morphology in accordance with one embodiment of the invention.

FIG. 21A-21B illustrate a schematic showing impingement of densely packed surface features in accordance with one embodiment of the invention.

FIG. 22A-22H illustrate mold designs and wet embossed samples with aesthetic patterns in accordance with one embodiment of the invention.

### DETAILED DESCRIPTION

Provided herein are mycelium materials and composite mycelium materials with favorable mechanical and aesthetic properties, and methods of producing such materials. Composite materials formed from mycelium biomass may tend to be relatively stiff and inflexible, and lack the desired strength required for various material applications. Surprisingly, it has been discovered that adding deformation areas to mycelium materials can improve the flexibility and strength of the materials, such as by reducing flexural modulus. In one aspect of the invention, the deformation areas are discrete deformation areas. In one aspect of the invention, the deformation areas are continuous deformation areas.

The materials described herein generally are formed from mycelium biomass, then wetlaid and dried into crust material. The crust material can be finished based on the end-use application of the material. Exemplary patents and applications discussing methods of growing mycelium include but are not limited to: WIPO Patent Publication No. 1999/024555; G.B. U.S. Pat. No. 2,148,959; G.B. Patent No. 2,165,865; U.S. Pat. Nos. 5,854,056; 2,850,841; 3,616,246; 9,485,917; 9,879,219; 9,469,838; 9,914,906; 9,555,395; U.S. Patent Publication No. 2015/0101509; U.S. Patent Publication No. 2015/0033620, all of which are incorporated herein by reference in their entirety. U.S. Pat. No. 11,015,059 describes composite mycelium material and methods for production of such material, and U.S. Patent Application No. 2018/0282529, discusses various mechanisms of solution-based post-processing mycelium material, each incorporated by reference in their entirety.

As described herein, the mycelium fibers, along with any suitable additives, can be formed into and slurry and wetlaid onto a mold such that structures are embossed in the material during processing and drying. The structures can protrude from a surface of the material and can be further compressed flat to create disruption areas in the material. Such disruption areas include fibers that are deformed, such as folded, undulated, bent, or otherwise oriented in the z-direction. In addition, in some instances the disruption areas can be denser than the surrounding area and can strengthen the material in the disruption area. Generally, the disruption areas do not extend through the thickness of the material, leaving a relatively smooth surface on the side of the material that is not embossed. It is hypothesized that flexural modulus is locally decreased where there is a disruption area, which increases the overall flexibility of the material. In addition, where the material has non-uniform thickness and density (even if the weight of the entire material is constant) having thinner areas with locally decreased flexural modulus also can increase the flexibility of the entire material.

The details of various embodiments of the disclosure are set forth in the description below. Other features, objects, and advantages of the disclosure will be apparent from the description. Unless otherwise defined herein, scientific and technical terms used in connection with the present disclosure shall have the meanings that are commonly understood by those of ordinary skill in the art.

### Definitions

The following terms, unless otherwise indicated, shall be understood to have the following meanings:

The term “mycelium” refers to a structure formed by one or more fibers. Mycelium is a distinct and separate structure from a fruiting body of a fungus or sporocarp.

The terms “cultivate” and “cultivated” refer to the use of defined techniques to deliberately grow a fungus or other organism.

The term “cultivated mycelium material” or “mycelium material” refers to material that includes one or more masses of cultivated mycelium, or includes solely of cultivated mycelium. In some embodiments, the one or more masses of cultivated mycelium is disrupted as described herein. In most cases, the cultivated mycelium material has been generated on a solid or liquid substrate, as described below.

The term “composite mycelium material” refers to any material including cultivated mycelium material combined with another material, such as a bonding agent or a supporting material as described herein, such as a crosslinking agent, natural adhesive, or a synthetic adhesive. In some embodiments, the mycelium comprises a supporting material. Suitable supporting materials include, but are not limited to, a mass of contiguous, disordered fibers (e.g. non-woven fibers), a perforated material (e.g. metal mesh, perforated plastic), a mass of discontiguous particles (e.g. pieces of woodchip) or any combination thereof. In specific embodiments, the supporting material is selected from the group consisting of a mesh, a cheesecloth, a fabric, a knit, a woven, and a non-woven textile. In some embodiments, the mycelium comprises a reinforcing material. A reinforcing material is a supporting material that is entangled within a mycelium or composite mycelium material. In some embodiments, the mycelium comprises a base material. A base material is a supporting material that is positioned on one or more surfaces of the mycelium or composite mycelium material.

The term “continuous” when referring to a pattern or disruption area, refers to a structure that extends across the material surface in the x-y plane, such as a structure that is curvilinear, or a structure having interconnected or intersecting elements. In some embodiments, continuous disruption areas can be connected to each other in one or more directions and are generally bordered by non-disrupted material on two or more sides, such as on three or more sides, or four or more sides. For example, in some embodiments, continuous disruption areas can be quadrilateral-shaped and connected to each other at the corners, and the four sides of the disruption areas are bordered by non-disrupted material. As such, in some embodiments, the disruption areas are formed in a continuous pattern and the continuous pattern is a geometric repeating, non-random continuous pattern.

The term “disruption area” refers to an area of the material that is deformed. The material is deformed in the disruption area by displacement of fibers from the typical longitudinal orientation in the x-y plane. The displaced fibers can be orientated in a transverse orientation in the z-direction after deformation, and can be formed into undulations, folds, compressions, or otherwise oriented in the z-direction. The deformed fibers can be formed into any suitable structure or shape, such as an undulation, a rivet, a mushroom, or an anvil, or any other suitable shape.

The term “discrete” means distinct or unconnected. Discrete disruption areas are therefore visually distinct or unconnected from each other in all directions. Such discrete disruption areas can be surrounded by non-disrupted material. In some embodiments, discrete disruption areas can be created using a mold that has a pattern of disconnected repeating shapes, such as a mold with a plurality of holes.

The term “incorporate” refers to any substance, e.g., cultivated mycelium material, composite mycelium material, or a bonding agent, that can be combined with or contacted with another substance. In a specific embodiment, a mycelium or composite mycelium material can be combined with, contacted with, or incorporated into a supporting material, e.g., woven, twisted, wound, folded, entwined, entangled, or braided together, to produce a mycelium material that has become incorporated with the supporting material. In another embodiment, one or more bonding agents may be incorporated within the cultivated mycelium material to be bonded, either in its disrupted or undisrupted state, e.g., embedded throughout the material, or added as a thin coating layer, such as by spraying, saturation, dipping, nip rolling, coating, and the like, to produce a mycelium material.

As used herein, the term “disrupted” or “manipulate” with respect to one or more fibers refer to one or more fibers of which one or more disruptions have been applied. A “disruption” or “manipulation” as described herein, may be mechanical or chemical, or a combination thereof. In some embodiments, the one or more fibers is disrupted by a mechanical action. A “mechanical action” as used herein refers to a manipulation of or relating to machinery or tools. Exemplary mechanical actions include, but are not limited to, blending, chopping, impacting, compacting, bounding, shredding, grinding, compressing, high-pressure, shearing, laser cutting, hammer milling, needling, needle felting, needle punching, and waterjet forces. In some embodiments, a mechanical action may include applying a physical force, e.g., in one or more directions such that the at least some of the fibers are aligned in parallel in one or more directions, wherein the physical force is applied repeatedly. In some other embodiments, the one or more fibers is disrupted by chemical treatment. “Chemical treatment” as used herein refers to contacting the mycelium material or composite mycelium material with a chemical agent, e.g., a base or other chemical agent, in an amount sufficient to cause a disruption. In various embodiments, a combination of mechanical actions and chemical treatments may be used herein. The amount of mechanical action (for example, the amount of pressure) and/or chemical agent applied, the period of time for which the mechanical action and/or chemical treatment is applied, and the temperature at which the mechanical action and/or chemical agent is applied, depends, in part, on the components of the cultivated mycelium material or composite mycelium material, and are selected to provide an optimal disruption on the cultivated mycelium material or composite mycelium material.

The term “mold” as used herein refers to a three-dimensional patterned surface that allows fluid flow, such as a mold, embossing roll configuration, plate, belt, or other forming surface. The topography and geometric design of the patterned surface can vary depending on the desired pattern of the resulting material. The mold is typically designed to be pervious to fluid to facilitate drying of the material.

The term “plasticizer” as used herein refers to any molecule that interacts with a structure to increase mobility of the structure.

The term “processed mycelium material” as used herein refers to a mycelium that has been post-processed by any combination of treatments with preserving agents, plasticizers, finishing agents, dyes, and/or protein treatments.

The term “web” as used herein refers to a mycelium material or composite mycelium material that has been disrupted, converted into a slurry, and arranged in a formation (e.g. drylaid, airlaid and/or wetlaid).

The term “z-direction” as used herein refers to fibers disposed outside of the plane of orientation of a web or panel. The term “is oriented in a z-direction” as used herein refers to fibers that become oriented during forming of a mycelium web resulting from processing of a composite mycelium material.

The term “fibrous” as used herein refers to any combination of pulp fibers, synthetic fibers, natural fibers, biocomponent fibers, continuous filaments or mixtures thereof.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated by reference to disclose and describe the materials and/or methods in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range, is encompassed within the aspects of the present disclosure. The upper and lower limits of these smaller ranges may independently be included in the smaller ranges, and are also encompassed within the aspects of the present disclosure, subject to any specifically excluded limit in the stated range. Where the stated range includes one or both of the limits, ranges excluding either or both of those included limits are also included in the aspects of the present disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Exemplary methods and materials are described below, although methods and materials similar or equivalent to those described herein can also be used in the practice of the present disclosure and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Mycelium Compositions and Methods of Production

Provided herein are mycelium materials and composite mycelium materials. The mycelium materials include mycelium fibers. In some or most embodiments, the mycelium materials are planar and have a first surface, a second surface opposite the first surface, and a thickness. In some or most embodiments, the first surface has one or more deformation areas spaced apart from each other. In some embodiments, the one or more deformation areas do not extend from the first surface to the second surface. In some or most embodiments, the mycelium material has a first region having a first density, and a second region having a deformation area extending from the first surface toward the second surface. In some embodiments, the second region has a second density that is higher than the first density. Methods of producing mycelium materials also are provided.

Provided herein, according to some embodiments, are various mycelium materials and methods for production thereof to provide mycelium materials and composite mycelium materials with favorable mechanical and aesthetic qualities, and related advantages.

In one aspect, provided herein are mycelium materials comprising mycelium fibers. In one aspect, provided herein are mycelium materials comprising mycelium fibers, the mycelium material is planar and has a first surface, a second surface opposite the first surface, and a thickness. The first surface comprises one or more deformation areas spaced apart from each other, and the one or more deformation areas do not extend from the first surface to the second surface.

Also provided herein are mycelium materials comprising mycelium fibers, where the mycelium material is planar and has a first surface, a second surface opposite the first surface, and a thickness. The first surface comprises one or more discrete deformation areas spaced apart from each other, and the one or more discrete deformation areas do not extend from the first surface to the second surface.

In one aspect, provided herein are mycelium materials comprising mycelium fibers, where the mycelium material is planar and has a first surface, a second surface opposite the first surface, and a thickness. The first surface comprises one or more continuous deformation areas spaced apart from each other, and the one or more continuous deformation areas do not extend from the first surface to the second surface.

Further provided herein are mycelium materials comprising mycelium fibers, where the mycelium material is planar and has a first surface, a second surface opposite the first surface, and a thickness. The first surface comprises one or more deformation areas spaced apart from each other, and the one or more deformation areas do not extend from the first surface to the second surface and the one or more deformation areas have a higher density than surrounding areas.

Also provided herein are mycelium materials comprising mycelium fibers, where the mycelium material is planar and has a first surface, a second surface opposite the first surface, and a thickness. The first surface comprises one or more deformation areas spaced apart from each other, and the one or more deformation areas do not extend from the first surface to the second surface and a majority of the mycelium fibers are orientated in an x-y plane and the one or more deformation areas comprise one or more disrupted fibers that are oriented in a z-direction.

In one aspect, provided herein are mycelium materials that are a composite mycelium material comprising a bonding agent and an additive.

In another aspect, provided herein are mycelium materials that are a composite mycelium material comprising a bonding agent and an additive, where the bonding agent comprises an adhesive, a resin, a crosslinking agent, and/or a matrix.

In one aspect, provided herein are mycelium materials that are a composite mycelium material comprising a bonding agent and an additive, where the bonding agent is selected from the group consisting of a vinyl acetate-ethylene (VAE) copolymer, a vinyl acetate-acrylic copolymer, a polyamide-epichlorohydrin resin (PAE), a copolymer, transglutaminase, citric acid, genipin, alginate, gum arabic, latex, a natural adhesive, and a synthetic adhesive.

In one aspect, provided herein are mycelium materials that comprise an additive.

In one aspect, provided herein are mycelium materials that comprise an additive that comprises lyocell fiber or an abaca fiber.

In one aspect, provided herein are mycelium materials comprising mycelium fibers, where the mycelium material is planar and has a first surface, a second surface opposite the first surface, and a thickness. The first surface comprises one or more deformation areas spaced apart from each other, and the one or more deformation areas do not extend from the first surface to the second surface and a majority of the mycelium fibers are orientated in an x-y plane and the one or more deformation areas comprise one or more disrupted fibers that are oriented in a z-direction and the one or more disrupted fibers have a length of 0.1 mm to 5 mm.

In another aspect, provided herein are mycelium materials comprising mycelium fibers. The mycelium material is planar and has a first surface, a second surface opposite the first surface, and a thickness. The mycelium material has a first region having a first density, and a second region having a deformation area extending from the first surface toward the second surface, and the second region has a second density that is higher than the first density.

In one aspect, provided herein are mycelium materials comprising mycelium fibers. The mycelium material is planar and has a first surface, a second surface opposite the first surface, and a thickness. The mycelium material has a first region having a first density, and a second region having a deformation area extending from the first surface toward the second surface, and the second region has a second density that is higher than the first density and the mycelium material has a plurality of deformation areas and a deformed volume fraction of less than about 50% of the mycelium material, less than about 40% of the mycelium material, less than about 30% of the mycelium material, or less than about 20% of the mycelium material.

In one aspect, provided herein are mycelium materials where the deformation areas are discrete deformation areas.

In another aspect, provided herein are mycelium materials where the deformation areas are continuous deformation areas.

In one aspect, provided herein are mycelium materials where the deformation areas include a plurality of first deformation areas that are discrete deformation areas and a plurality of second deformation areas that are continuous deformation areas.

In another aspect, provided herein is a method of producing a mycelium material. The method comprises providing mycelium fibers, adding a solution to the mycelium fibers to produce a mycelium composition, wet embossing the mycelium composition such that one or more deformation areas are formed, and drying the mycelium composition to produce the mycelium material.

In a further aspect, provided herein is a method of producing a mycelium material. The method comprises providing mycelium fibers, adding a solution to the mycelium fibers to produce a mycelium composition, adding a bonding agent to the mycelium composition, wet embossing the mycelium composition such that one or more deformation areas are formed, and drying the mycelium composition to produce the mycelium material.

In one aspect, provided herein is a method of producing a mycelium material. The method comprises providing mycelium fibers, adding a solution to the mycelium fibers to produce a mycelium composition, wet embossing the mycelium composition such that one or more deformation areas are formed, and drying the mycelium composition to produce the mycelium material. The mycelium material is planar and has a first surface, a second surface opposite the first surface, and a thickness, and the one or more deformation areas are spaced apart from each other and do not extend from the first surface to the second surface.

In one aspect, provided herein is a method of producing a mycelium material. The method comprises providing mycelium fibers, adding a solution to the mycelium fibers to produce a mycelium composition, wet embossing the mycelium composition such that one or more deformation areas are formed, and drying the mycelium composition to produce the mycelium material. The mycelium material is planar and has a first surface, a second surface opposite the first surface, and a thickness, and the one or more deformation areas are spaced apart from each other and do not extend from the first surface to the second surface and a majority of the mycelium fibers are orientated in an x-y plane and the one or more deformation areas comprise one or more disrupted fibers that are oriented in a z-direction.

In one aspect, provided herein is a method of producing a mycelium material with deformation areas wherein the deformation areas are discrete deformation areas.

In another aspect, provided herein is a method of producing mycelium materials with deformation areas wherein the deformation areas are continuous deformation areas.

In one aspect, provided herein is a method of producing mycelium materials with deformation areas wherein the deformation areas include a plurality of first deformation areas that are discrete deformation areas and a plurality of second deformation areas that are continuous deformation areas.

In some embodiments, the mycelium material is a composite mycelium material comprising a reinforcing material. In some embodiments, the reinforcing material is entangled within the composite mycelium material. In some embodiments, the reinforcing material comprises a base material. In some embodiments, the base material is positioned on one surface of the composite mycelium material. In some embodiments, the base material is positioned within the composite mycelium material. In some embodiments, the reinforcing material is selected from the group consisting of a mesh, a cheesecloth, a fabric, a knit fabric, a woven fabric, and a non-woven fabric. In some embodiments, the reinforcing material is entangled within the composite mycelium material. In some embodiments, the reinforcing material comprises a base material. In some embodiments, the base material is positioned on one surface of the composite mycelium material. In some embodiments, the base material is positioned within the composite mycelium material. In some embodiments, the reinforcing material is selected from the group consisting of a mesh, a cheesecloth, a fabric, a knit fabric, a woven fabric, and a non-woven fabric.

In some embodiments, the mycelium material further comprises a dye. In some embodiments, the method comprises further adding a dye. In some embodiments, the dye is selected from the group consisting of an acid dye, a direct dye, a synthetic dye, a natural dye, and a reactive dye. In one aspect, the dye is a reactive dye. In some aspects, the composite mycelium material is colored with the dye and the color of the composite mycelium material is substantially uniform on one or more surfaces of the composite mycelium material. In some embodiments, the method comprises further coloring the composite mycelium material with the dye, wherein the color of the composite mycelium material is substantially uniform on one or more surfaces of the composite mycelium material. In other aspects, the dye is present throughout the interior of the composite mycelium material.

In some embodiments, the mycelium material further comprises a plasticizer. In one aspect, the method comprises further adding a plasticizer. In some embodiments, the plasticizer is selected from the group consisting of oil, glycerin, fatliquor, sorbitol, diethyloxyester dimethyl ammonium chloride, Tween 20, Tween 80, m-erythritol, water, glycol, triethyl citrate, water, acetylated monoglycerides, epoxidized soybean oil, aliphatic chain compound, 2-octenyl succinic anhydride (OSA), 2-dodecenyl succinic anhydride, octacedenyl succinic anhydride, stearic anhydride, 3-Chloro-2-hydroxypropyldimethyldodecylammonium chloride, heptanoic anhydride, butyric anhydride, chlorohydrin, and siloxane. In some embodiments, the composite mycelium material is flexible.

In some embodiments, the mycelium material further comprises an additive. In one aspect the additive comprises a lyocell fiber. In another aspect, the additive comprises an abaca fiber.

In some embodiments, the mycelium material comprises a mechanical property. In some embodiments, the mechanical property comprises a wet tensile strength, an initial modulus, an elongation percentage at the break, a thickness, a slit tear strength, and/or a peel resistance. In one aspect, the peel resistance comprises an average T peel value of 5-20 N/cm and a max T peel value of 10-30 N/cm.

In some embodiments, the one or more fibers are disrupted by a mechanical action. In some embodiments, the mechanical action comprises blending the one or more fibers. In some embodiments, the one or more fibers is disrupted by chemical treatment. In some embodiments, the chemical treatment comprises contacting the one or more fibers with a base or other chemical agent in an amount sufficient to cause a disruption. In some embodiments, the base comprises alkaline peroxide.

In some embodiments, the mycelium composition comprises a solid content of 1-10%. In some embodiments, the mycelium composition comprises a solid content of 3-7%. In some embodiments, the mycelium composition comprises a solid content of 4-6%. In some embodiments, the mycelium composition comprises a solid content of 1-6%.

### Description of the Technology

Wet embossing is the process of wetlaying with a textured surface (referred to as a “mold,” embossing roll, plate, belt, or other forming surface) instead of a normal (flat) forming cloth. The goal is to impart the texture of the mold onto the web in order to affect aesthetic and/or mechanical properties. Molds are typically designed to create z-directional fiber orientation in the web, or otherwise disrupt the predominantly x/y plane fiber orientation achieved in a normal wetlay. Molds can also be designed to impart a specific aesthetic pattern or texture to the web. A typical mold will have features on the order of 2-10 mm. Examples of molds include shelf/drawer liners and custom 3D printed or laser cut arrays of pillars and/or holes.

It has been discovered that wet embossing mycelium materials can provide increased drape, dry tensile strength, elongation at break and T-peel force relative to non-wet embossed samples. In addition, wet embossing can impart desirable aesthetic patterns to the mycelium web. Because wet embossing can create materials with non-uniform density and surface smoothness, care must be taken to choose embossing patterns that do not interfere with finish adhesion or create unacceptable surface aesthetics.

Wet embossing typically happens over the entire dewatering process. The slurry is poured over the mold or the mold is applied to the slurry, which is left in until dewatering is complete. In wet emboss, the “thickness” of the disrupted volume of the web is determined by the height of the mold features. The amount of disrupted vs. undisrupted volume is hypothesized to affect the tensile strength of the material and the surface smoothness

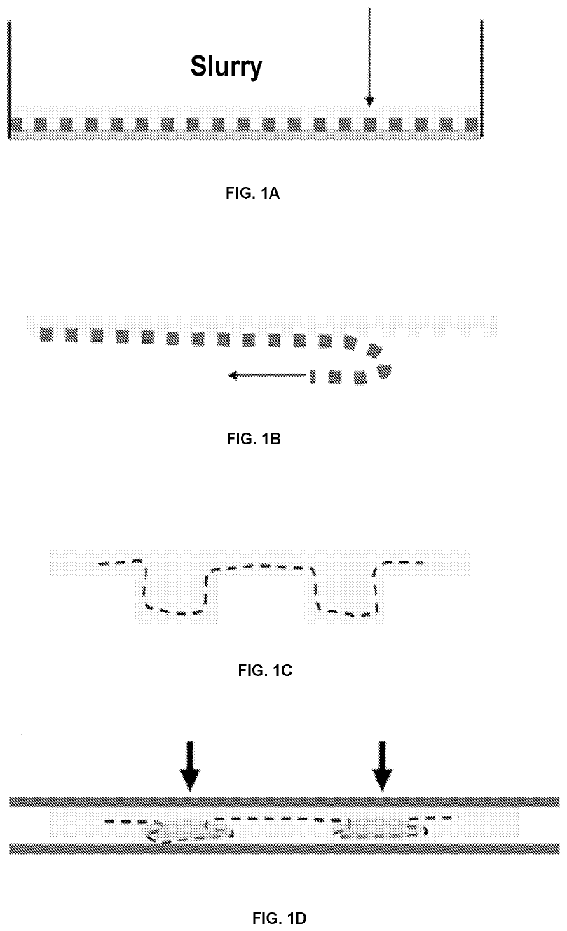

FIG. 1A-1D illustrates a schematic overview of the wet emboss process.

FIG. 1A shows slurry is wetlaid over a mold, which is placed on top of the forming cloth.

FIG. 1B shows after wetlay, the mold is removed from the web.

FIG. 1C shows the dry web has features corresponding to the negative of the mold, typically with a disruption area. As shown in FIG. 1C, the disruption area can have an undulating fiber orientation (represented by the dotted line).

FIG. 1D shows when the dry web is pressed, the features can further deform by upsetting, folding, or pleating.

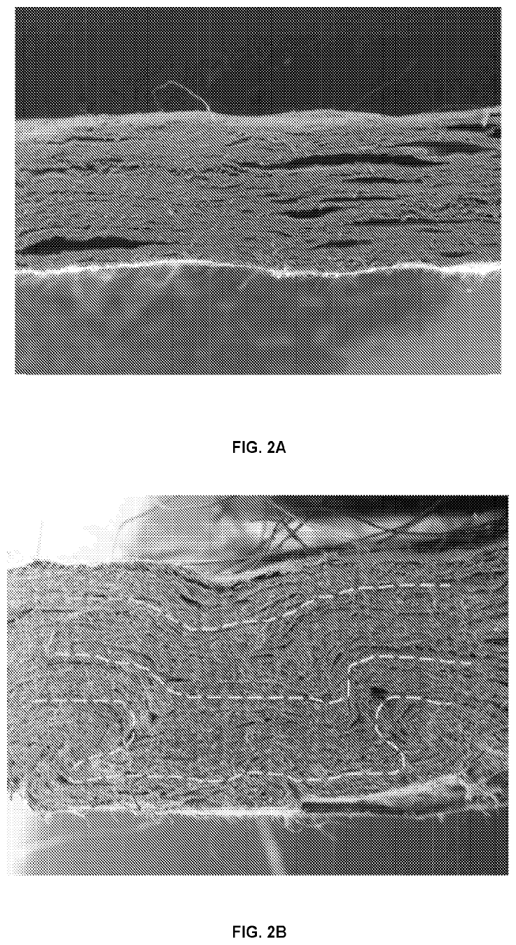

FIG. 2A-2B illustrate scanning electron micrographs

FIG. 2A shows an example of a cross sections of a non-embossed crust material.

FIG. 2B shows an example of a cross sections of a wet embossed crust material.

Dotted lines have been drawn in to highlight the fiber orientation throughout the wet embossed material. The embossed side is on the bottom of the images.

The standard sample shows predominantly in-plane (X-Y) fiber orientation, which is formed as the fibers lay on top of each other and parallel to the forming cloth during the wetlay process. By contrast, the wet emboss sample shows a deformation area with varying degrees of undulation. These structures are formed during the wetlay as the fibers lay over the features of the mold. Once dried, these structures can be further deformed by pressing.

Increased drape and peel resistance in wet embossed samples is due to the undulated fiber network formed during the wetlay. Wherever there is an undulation or fold in the structure, the flexural modulus is locally decreased. The overall flexibility of the material is therefore increased relative to a material with no undulations.

A secondary effect of wet embossing on drape is that depending on the mold used, the sample can have non-uniform thickness and density even if the weight of the entire sample is the same. Since flexural modulus is strongly proportional to thickness, having thinner areas with locally decreased flexural modulus can also increase the drape of the entire material.

Note that for the wet embossed sample shown in FIG. 2B, the degree of undulation is highest on the embossed surface (bottom of image) and decreases towards the top surface. This can be explained by two effects detailed below.

As the slurry lays into the mold, the fibers closest to the mold conform to the mold features with the highest fidelity. As more material builds up around the features over the course of dewatering, the fidelity to the pattern decreases. Molds can also be designed with short (relative to the wetlaid web thickness) features which intentionally minimize disruption to the non-embossed (top) surface. This is advantageous if a smooth top surface is desired for finishing. Depending on their shape, features on the embossed surface will deform further upon pressing. For example, a protruding pillar on a web may be upset upon pressing, with the top of the pillar squashing/folding over like the head of a rivet.

This relationship between the wetlay process, the degree of deformation such as undulation, and the flexural properties can also explain why wetlaying over an emboss mold may give different results than placing the mold on top of the slurry before wetlay. Three options for mold placement are described below. In the following schematics, mold features are shown as black shapes. Dotted lines indicate the fiber orientation in the web cross section after wetlay.

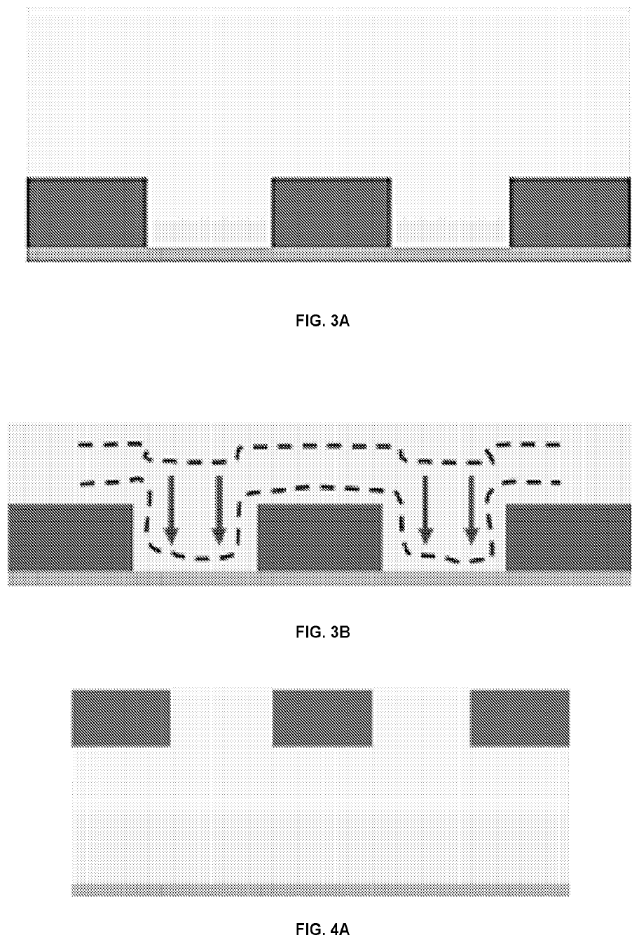

FIG. 3A-3B illustrate a schematic showing a mold placed on forming cloth, and the slurry is wetlaid over the mold as previously described. Slurry flows into mold and fibers deposit around mold features with high fidelity.

FIG. 3A shows the mold placed on top of the slurry before dewatering starts. The mold settles into the web as dewatering progresses.

FIG. 3B shows that the mold only starts to deform the web as dewatering is almost complete, i.e., when enough water is removed that deformations to the web are less reversible or non-reversible.

The majority of the wetlay resembles the standard non-embossed case, with most of the slurry not in contact with the mold and instead forming a standard web against the forming cloth. In some instances, the top of the web may show deformations, depending on the mold geometry. Where the degree of deformation such as undulation is expected to be low, the deformation areas likely will not extend throughout the thickness of the web and will not be visible at the top of the web disposed opposite the embossed side.

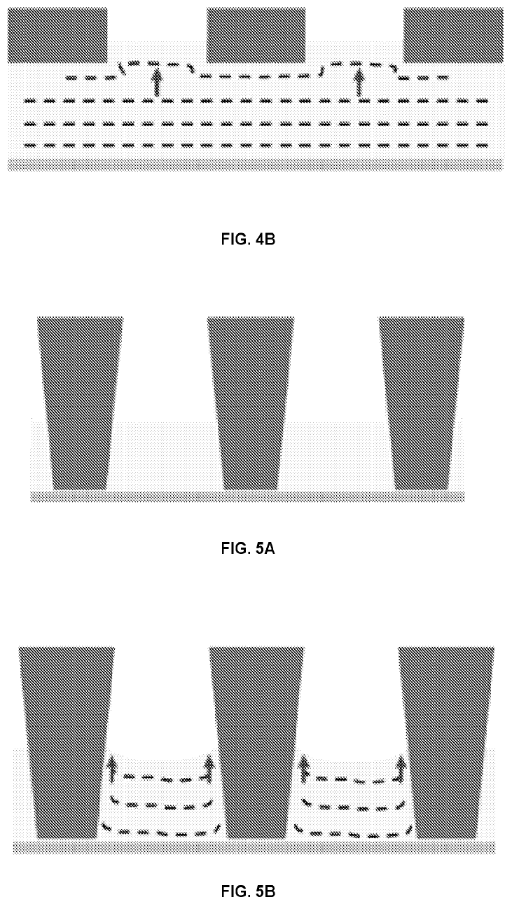

FIG. 4A-4B illustrate a schematic showing wetlay with a mold placed on top of the slurry.

FIG. 4A shows the mold positioned on top of the slurry.

FIG. 4B shows the position of the mold after partial dewatering.

In some embodiments, the mold can be pressed into the web after partial dewatering in order to impart more deformation to the web. The pressing can be done using any suitable method, including by pressing the web with one or more rollers or plates.

Applying the web to the top of the slurry is mechanistically distinct from the embodiment illustrated in FIG. 3A-B. In FIG. 3A-3B, feature formation starts when the water content of the slurry is the highest (i.e. the fibers are the most mobile and can easily conform to the mold), and then the features are “locked in place” as dewatering progresses.

In FIG. 4A-4B, feature formation starts at an intermediate water content and fiber mobility, and therefore the fidelity may be reduced. The effectiveness of feature formation by pressing the mold into a partially dewatered slurry will depend on the slurry viscosity and mold geometry (ex. small, sharp features will deform a dry/viscous slurry more easily than large, flat features). In some embodiments, the mold can be designed to provide enough deformation and thickness variation to the web to increase drape to an acceptable level, even if the degree of undulation in the deformation area is less than that obtained in the embodiment illustrated in FIG. 3A-B.

FIG. 5A-5B illustrate a schematic showing wetlay with a mold extending through the thickness of the slurry/web.

FIG. 5A shows slurry poured into the box having a mold with features tall enough to reach the forming cloth that is placed inside before dewatering. In this case, feature formation occurs by the slurry flowing around the features and slightly adhering to their sides as dewatering progresses. Between features, the slurry forms a predominantly undisrupted layer as shows in FIG. 4A-4B.

FIG. 5B shows that the result is a web with a mild degree of disruption in the areas adjacent to the mold, connected by a very thin layer of material trapped between the mold and the forming cloth. The final material will tend to have increased drape due to these thin areas, but these areas also are weak in tension and peel. However, it is possible for the embossed features (which now span almost the entire thickness of the web) to deform during pressing and increase drape and peel resistance.

In an alternative embodiment of the invention, the wet embossing is performed with the mold held several millimeters above the surface of the forming cloth, to allow a thicker undisrupted layer to form. Care must be taken to ensure the mold and the forming cloth remained parallel, and that the gap between them is consistent. One advantage of this approach is that the mold can be removed from the top relatively easily after dewatering. With this approach, the degree of undulation in the deformation area is typically reduced relative to the embodiments of the invention illustrated in the FIG. 3A-B wet embossing technique. The variation in web thickness typically provides increased drape relative to a standard wetlay.

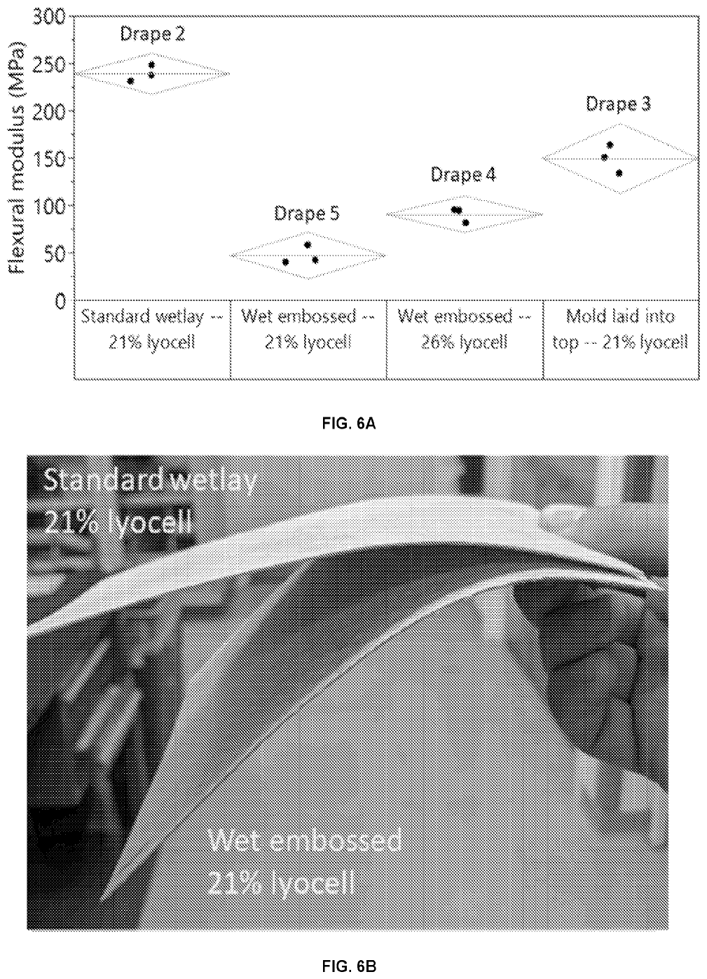

FIG. 6A-6B shows the flexural moduli and drape of samples made with and without wet embossing.

FIG. 6A show samples made in accordance with the techniques described in FIG. 3A-3B. The wet emboss had significantly lower flexural modulus than the standard sample, even when the 10 mm lyocell fiber input was increased over the baseline (26 vs 21%). Typically, increasing the lyocell input increases tear strength at the cost of reduced drape; however, this data illustrates how wet emboss can compensate for the drape while still allowing for higher fiber loading and higher tear resistance. In addition, the sample wetlaid with the mold laid into the top of the slurry (FIG. 4A-B) was significantly less flexible than either of the samples made with (FIG. 3A-B) wet emboss, although it was still more flexible than the standard sample. This demonstrates that even when using the same mold, how the emboss is applied can affect the mechanical properties. Data points represent individual measurements. Diamonds represent the mean and 95% confidence interval of the mean.

FIG. 6B shows visual comparison of a wet embossed sample produced in accordance with the technique described and illustrated in FIG. 3A-B compared to standard sample produced with no mold present during dewatering.

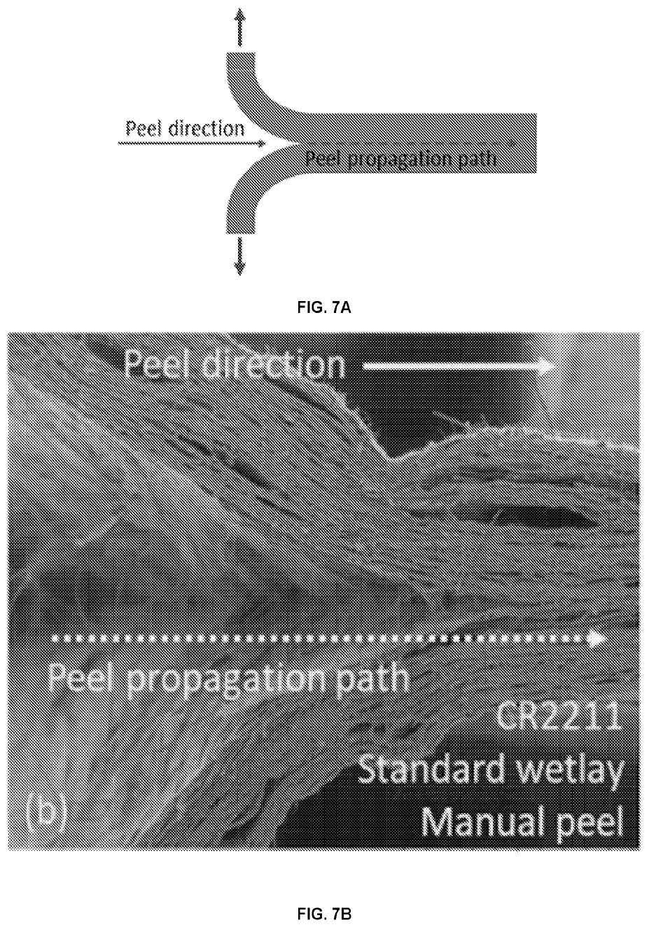

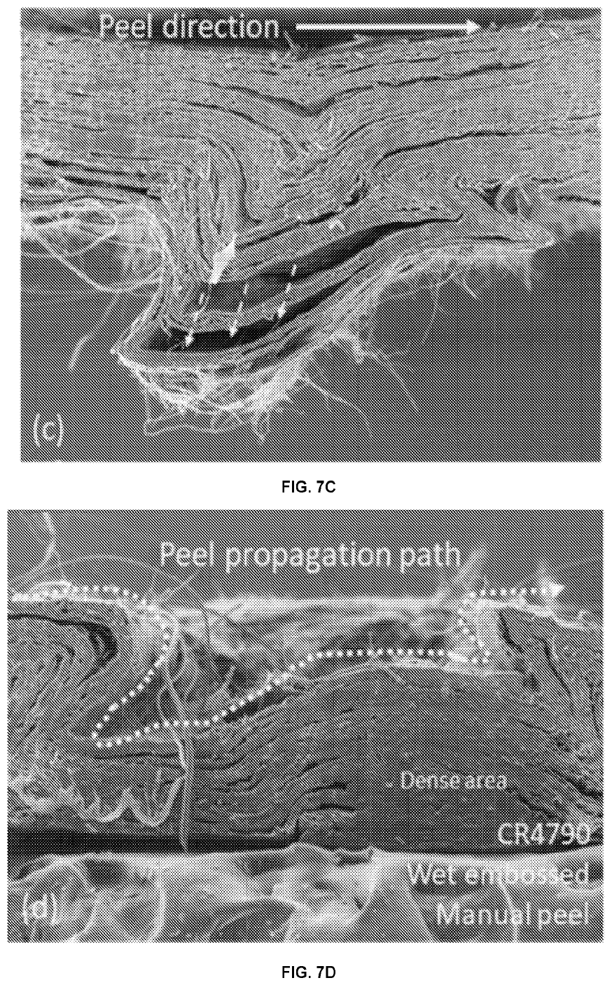

FIG. 7A-7D show a schematic and scanning electron micrographs of standard and wet embossed samples which were peeled apart manually to visualize the peel propagation path and extent of deformation.

FIG. 7A shows a schematic of a T-peel test. The sample is pulled apart in the vertical direction and the peel propagates in the horizontal direction.

FIG. 7B shows a cross section of a standard wetlay sample showing in-plane fiber orientation and peel propagation through the sample.

FIG. 7C shows a cross section of the top of a peeled wet emboss sample showing undulating fiber orientation and peel propagation path.

FIG. 7D shows a cross section of the bottom of a peeled wet emboss sample, showing undulating fiber orientation and peel propagation path.

Note also the delamination caused by the peel (dashed arrows in FIG. 7C) as the folded over feature was pulled out of the bottom, and how the peel propagation path avoids the dense area in FIG. 7D.

Wet emboss is hypothesized to increase peel resistance by inhibiting peel propagation by disrupted/undulating fiber orientation, as the peel is forced to take a tortuous path through the material. Indeed, highly undulated or folded over features, it may require extra force to unfold or peel a feature apart. In addition, depending on the emboss pattern, molded features may densify after pressing and these density variations in the deformation areas may also deflect or inhibit peel propagation. All three of the aforementioned mechanisms are suggested by the failure mode observed in FIGS. 7C and 7D. For reference, a standard sample is also shown in FIG. 7B, in which none of the three mechanisms are present.

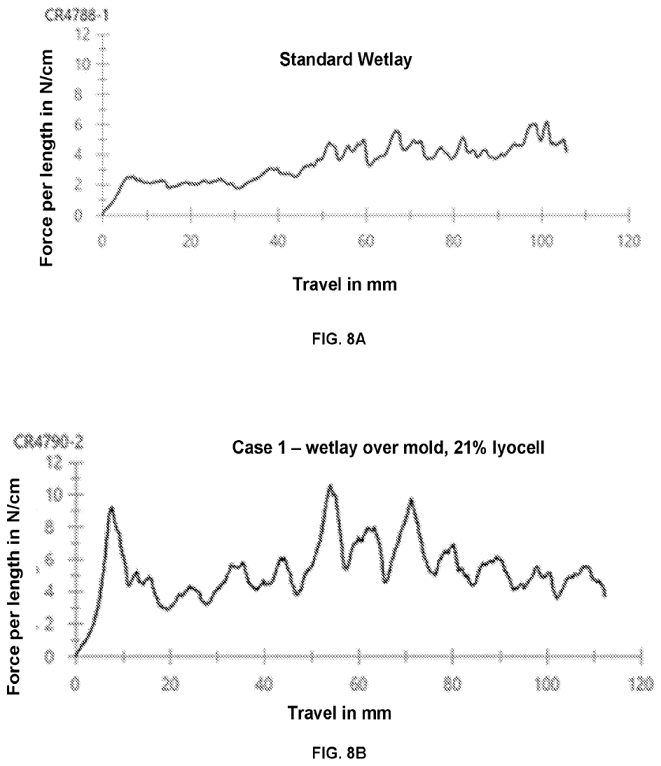

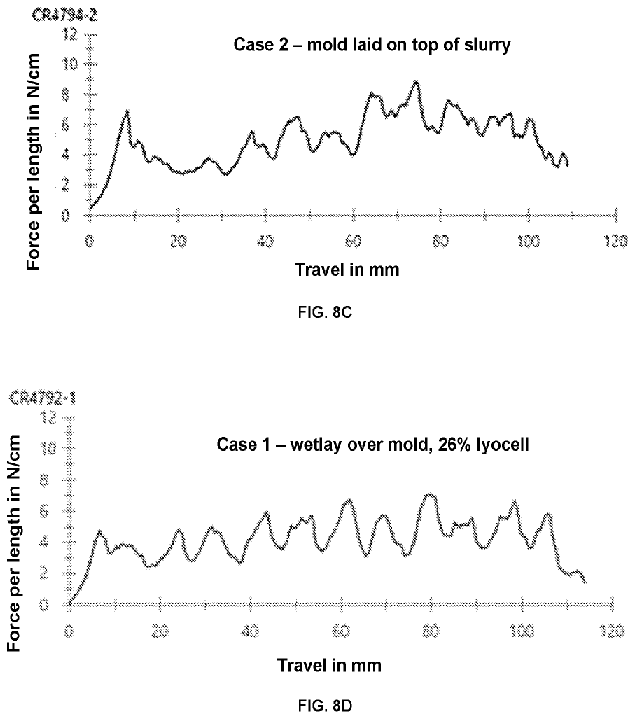

FIG. 8A-8D show representative force-travel plots from T-peel tests performed on standard and wet embossed samples.

FIG. 8A shows a standard wetlay sample that lacks these periodic peaks.

FIG. 8B shows a wet emboss with 21% lyocell produced in accordance with the techniques described in FIG. 3A-3B.

FIG. 8C shows a wet emboss with 21% lyocell produced in accordance with the techniques described in FIG. 4A-4B.

FIG. 8D shows a wet emboss with 26% lyocell produced in accordance with the techniques described in FIG. 3A-3B.

Each of the wet embossed samples show distinct periodic peaks in force, corresponding to individual embossed features (as in FIG. 7C and FIG. 7D) being pulled apart. Notably the standard wetlay sample lacks these periodic peaks, which demonstrates how wet embossing can measurably increase the peel resistance of a sample. In addition, the peaks in the wet emboss sample embodiments of the invention described in relation to FIGS. 4A-4B (and illustrated here in FIG. 8C) are less distinct than the wet emboss sample embodiments of the invention described in relation to FIGS. 3A-3B, suggesting that the features in the wet emboss sample embodiments of the invention described in relation to FIGS. 4A-4B are similarly less distinct.

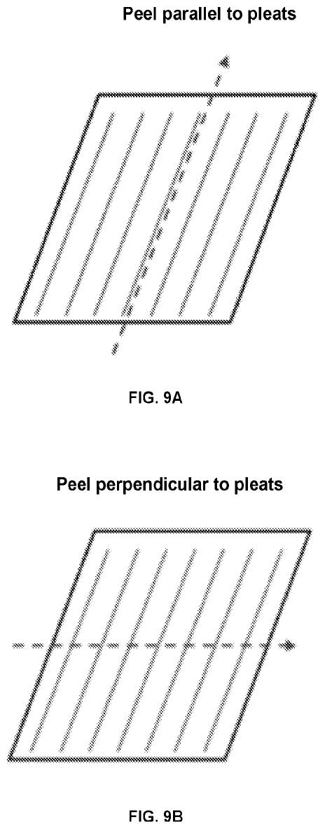

FIGS. 9A-9D show force-travel plots for embodiments of the invention described in relation to FIGS. 5A-5B wet emboss sample with unidirectional pleats. T-peel specimens were peeled either parallel or perpendicular to the pleat direction. These results further support the hypothesis that highly undulated emboss features of the disruption areas are the mechanism for increased peel resistance. In this experiment, a sample was prepared using the embodiment of the invention described in relation to FIGS. 5A-B. The sample was prepared using a mold consisting of closely spaced parallel slats running in the x-direction of the web. After pressing, the emboss features fold over and form a series of parallel pleats in the deformation areas.

FIGS. 9A and 9C show the force-travel plots for T-peel tests performed parallel to these pleats. When testing parallel along a pleat, the T-peel force is sustained and consistently high (average 8.1 N/cm).

FIGS. 9B and 9D show the force-travel plots for T-peel tests performed perpendicular to these pleats. It was found that, when testing perpendicular to the pleats, the force spikes when encountering a pleat and drops significantly when traveling between them, leading to a lower average force (3.6 N/cm).

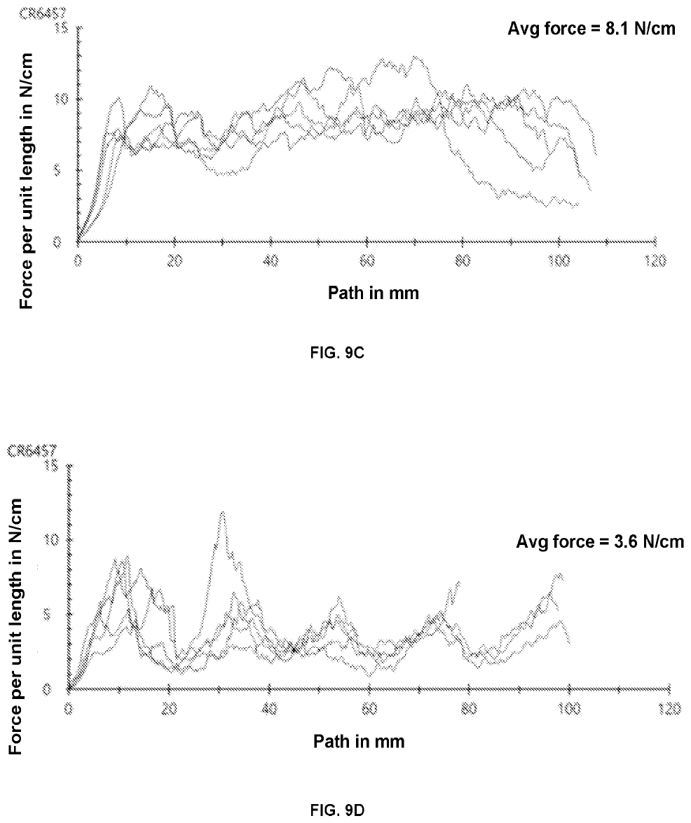

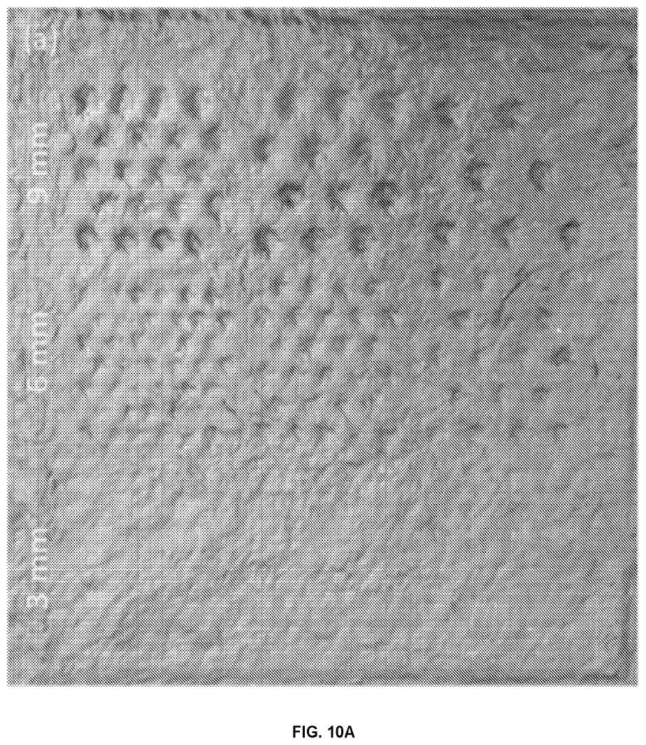

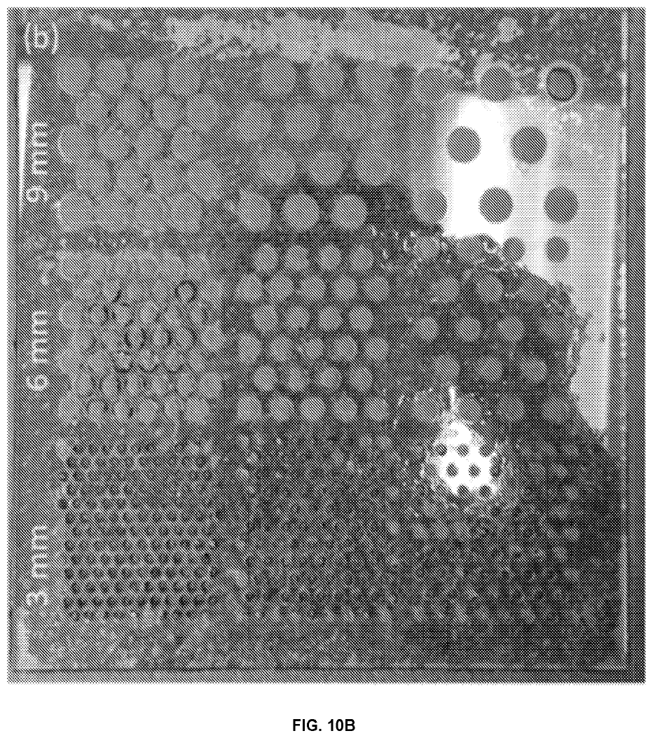

FIGS. 10A-10B show photographs of a web wetlaid into a 3.2 mm thick mold with arrays of 3 mm, 6 mm, and 9 mm diameter holes.

FIG. 10A shows a top surface of the web after wetlay.

FIG. 10B shows a bottom surface of the web/mold showing variable filling, particularly with the 3 mm diameter holes.

The results shown in FIGS. 10A-10B were conducted to investigate the effect of mold geometry on feature formation during wetlay.

It was found that effective feature formation depends on a combination of the slurry properties and mold geometry. How well the final web fills and conforms to the holes depends on the viscosity/surface tension of the slurry, the depth and diameter of the holes, and how vertical or angled the walls of the holes are. The ability of a slurry with a fixed composition to fill an array of holes with various dimensions was explored using laser cut acrylic molds that have holes with straight walls.

In addition to the variable filling as a function of hole diameter, the top surface of the web shows that as hole diameter increases, the top becomes more dimpled, and as hole diameter decreases, the top remains smooth. This demonstrates the deformed volume fraction; in other words, how much of the web is participating in the emboss versus how much is resting undisrupted on the surface. As more of the slurry is required to fill into large holes, there is not enough slurry to fill in the resulting dimple. Conversely, if the slurry does not fill into small holes, it is equivalent to a standard wetlay and the top surface remains undisrupted, which can provide a smooth surface for finishing.

The deformed volume fraction can be any suitable percentage of the web, such as a percentage of the web that provides a web with disruption areas and with a substantially smooth surface for finishing, such as about 40% or less of the web, about 35% or less of the web, about 30% or less of the web, about 25% or less of the web, about 20% or less of the web, about 15% or less of the web, about 10% or less of the web, or about 5% or less of the web.

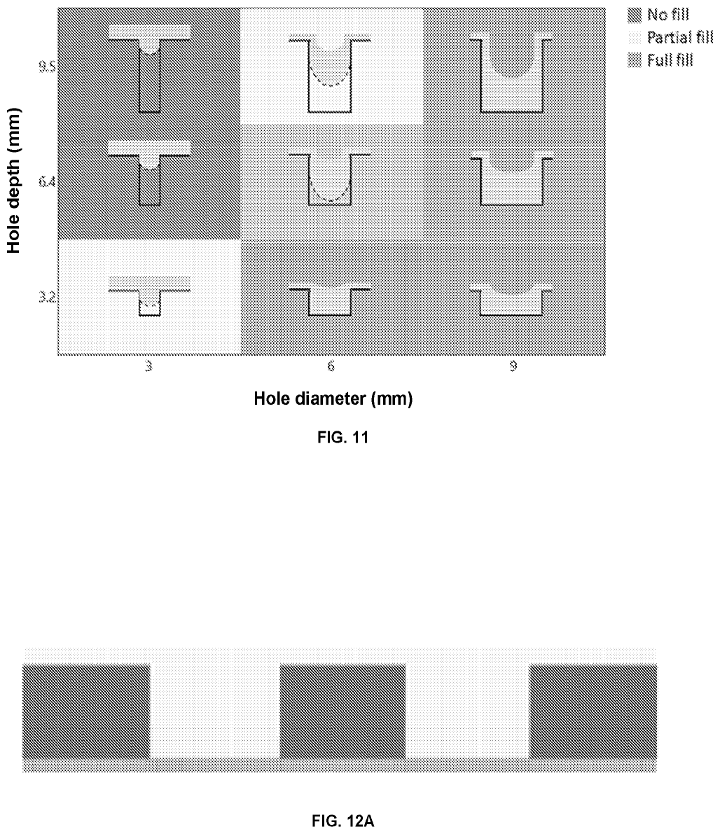

FIG. 11 shows a heatmap of feature forming ability as a function of (straight-walled) hole diameter and depth. For the baseline composition, features were successfully formed in the 6-9 mm ranges. Images are provided for visual reference only and are not drawn to scale.

The overall results are summarized in FIG. 11. In this experiment, the effect of hole spacing was also investigated but the effect was found to be minimal. At this composition, the slurry was largely unable to fill the 3 mm diameter holes. As the hole diameter increased to 6 and 9 mm, feature formation was more successful, albeit with increased dimpling as the hole depth increased.

It is hypothesized that water content (which decreases over the course of the wetlay) and the fiber composition of the slurry affects its surface tension and viscosity. A very dilute slurry or a slurry with no long fibers should fill in a given hole more easily than a slurry that is highly concentrated or contains a high fiber loading.

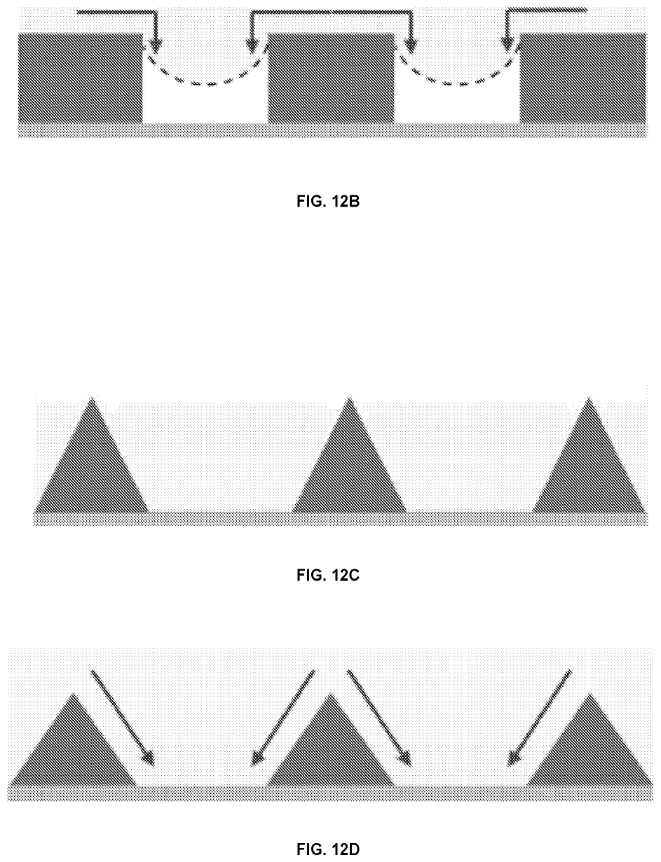

FIG. 12A-12D shows the effect of drafted mold features on slurry infiltration.

FIGS. 12A and 12C show molds with straight-walled features. Depending on the aspect ratio of the holes, the slurry may not be able to effectively fill them.

FIGS. 12B and 12D show molds with features of the same spacing, base area, and height as in FIGS. 12A and 12C, but with slanted walls. This increases the effective hole diameter and volume of the hole, allowing the slurry to fill in the entire hole more easily.

3D printed pillar array molds were explored to demonstrate that the inverse geometry of a laser cut hole array would not have periodic T-peel force (see FIG. 8A-8D). An additional advantage of 3D printing is that features are not restricted to having straight sides. Drafting or chamfering the mold features allows the mold to be removed more easily from the web, but also allows the negative space around them to be filled in more easily by the slurry.

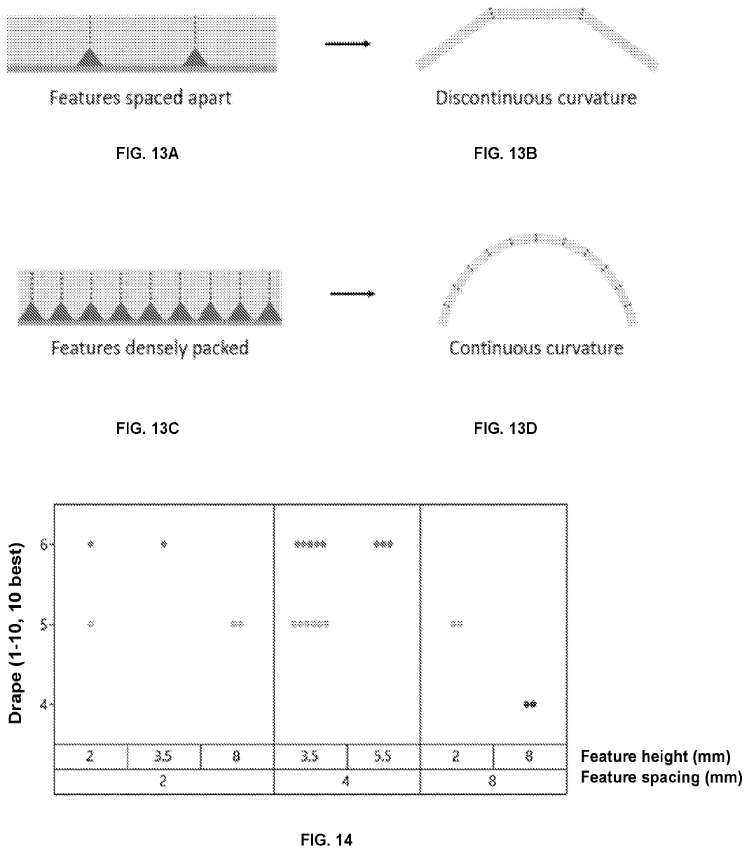

FIG. 13A-13D show a schematic showing effect of feature spacing on drape and the effect of mold geometry on drape.

FIG. 13A shows features spaced apart that give rise to a discontinuous curvature as shown in FIG. 13C.

FIG. 13B shows features densely packed that give rise to a continuous curvature as shown in FIG. 13D.

There are two aspects of drape which are affected by the mold geometry. The first is the absolute value of the drape (analogous to the flexural modulus), and the second is whether the material forms a smooth, continuous curve when flexed, or only bends at distinct points.

FIG. 14 shows drape grades for samples embossed with 3D printed pillar array molds with a range of feature heights and spacings. Points represent a single crust material.

The drape grades for samples embossed with 3D printed pillar array molds are shown. The samples are plotted as a function of mold feature spacing and height. As mold feature height and spacing varied from 2-8 mm, most samples achieved drape grades of 5-6. The samples with the worst drape were those with features spaced the farthest apart, i.e., with the greatest distance between bending points and consequently discontinuous curvature. The effect of feature size is analogous to feature spacing in that as feature size or spacing increases, the distance between bending points increases and the drape is expected to decrease. Thus, for a given input composition, the drape varies within a narrow range until feature spacing or size increases to become a large enough fraction of the bending circumference, at which point discontinuity in the bending curve is clearly visible.

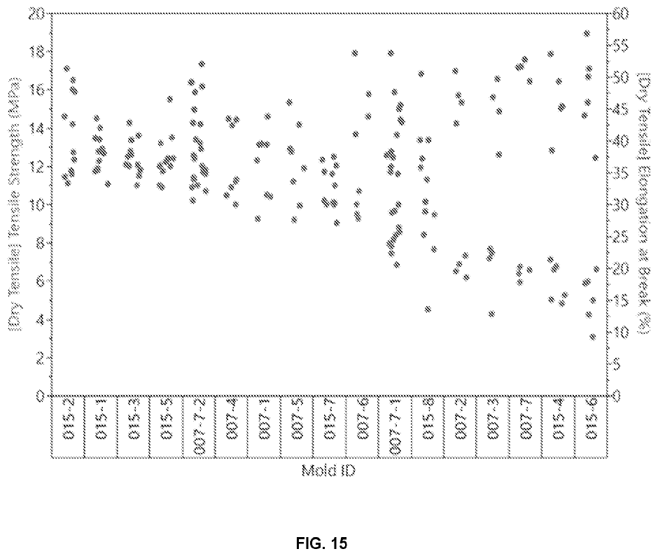

FIG. 15 plots dry tensile strength and elongation at break of samples for each mold used. The x-axis is ordered by decreasing tensile strength. Points represent individual specimens. For these samples, tensile strength is the most strongly affected mechanical property.

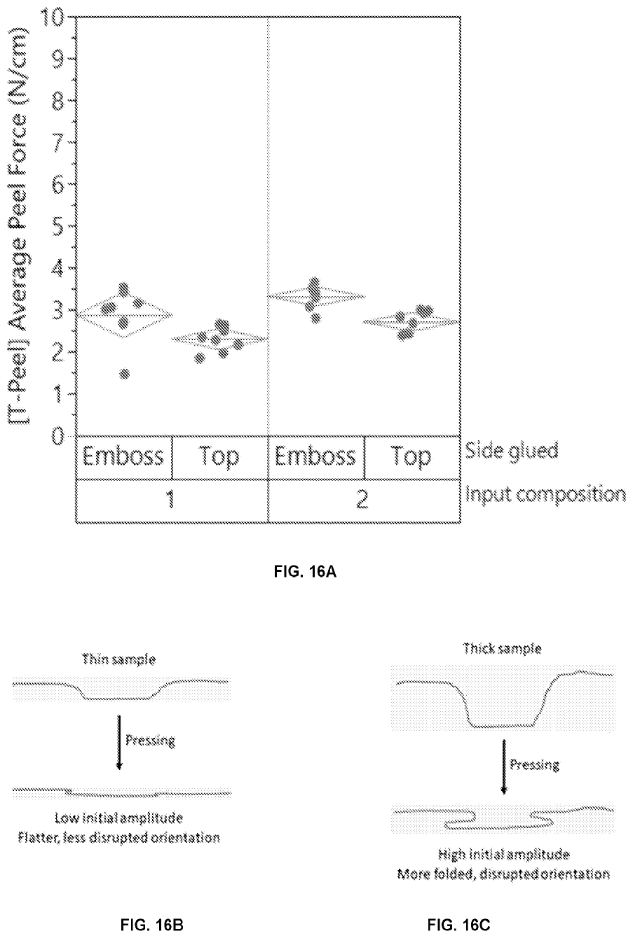

FIG. 16A-16C show average T-peel force for wet embossed thin samples having an average final thickness of 0.3 mm-0.4 mm. FIG. 16A and schematics FIG. 16B-16C illustrate how the maximum degree of undulation may depend on sample thickness. Wet embossing appears to be less effective for thin samples, since the maximum amplitude of an undulation is limited by the sample thickness. This reduces the degree to which an embossed feature can be further deformed upon pressing. Overall, this appears to reduce peel resistance.

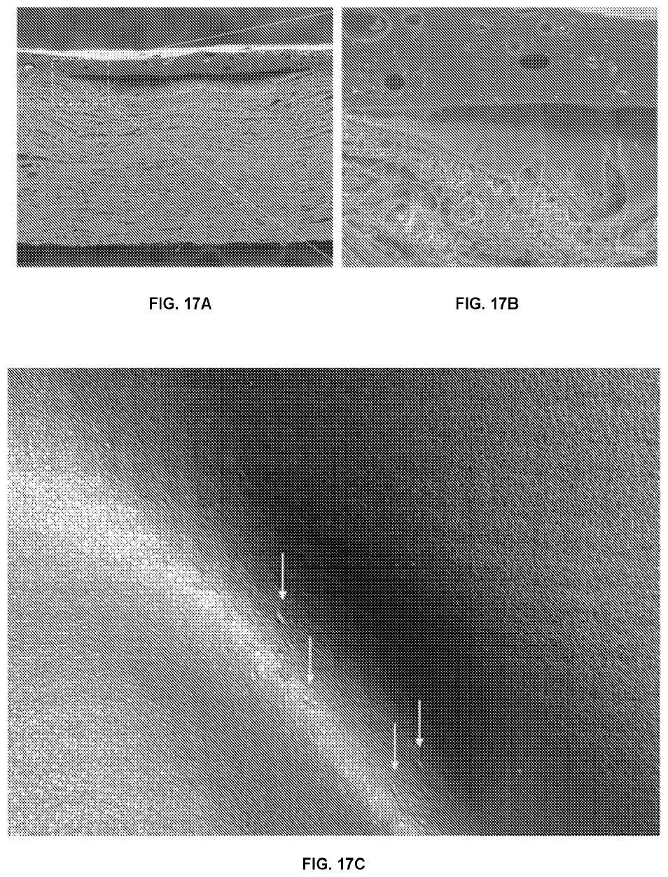

FIG. 17A-17C illustrate scanning electron micrographs of a bridge bubble formed on a sample with the finish layer applied to the embossed side. FIG. 17A-17B show incomplete coating of the adhesive layer on the sample beneath the “bridge”. FIG. 17C is a photograph of a wet embossed sample finished on the top surface, showing small bridge bubbles puckering out of the material as it is flexed. As shown in FIGS. 17A-17C, mold feature geometry can affect top surface smoothness.

Certain finishing approaches require a smooth, uniform surface comparable to the surface of a standard wetlay sample. The wet embossing process and resulting surface morphology can affect finishing. For example, if the surface has deep pits or valleys, the adhesive layer will not evenly coat the surface, resulting in the finish layer bridging the pit or valley without actually contacting the underlying substrate. This can result in aesthetically undesirable “bridge bubbles”, where the finish layer puckers away from the surface when the material is flexed inward.

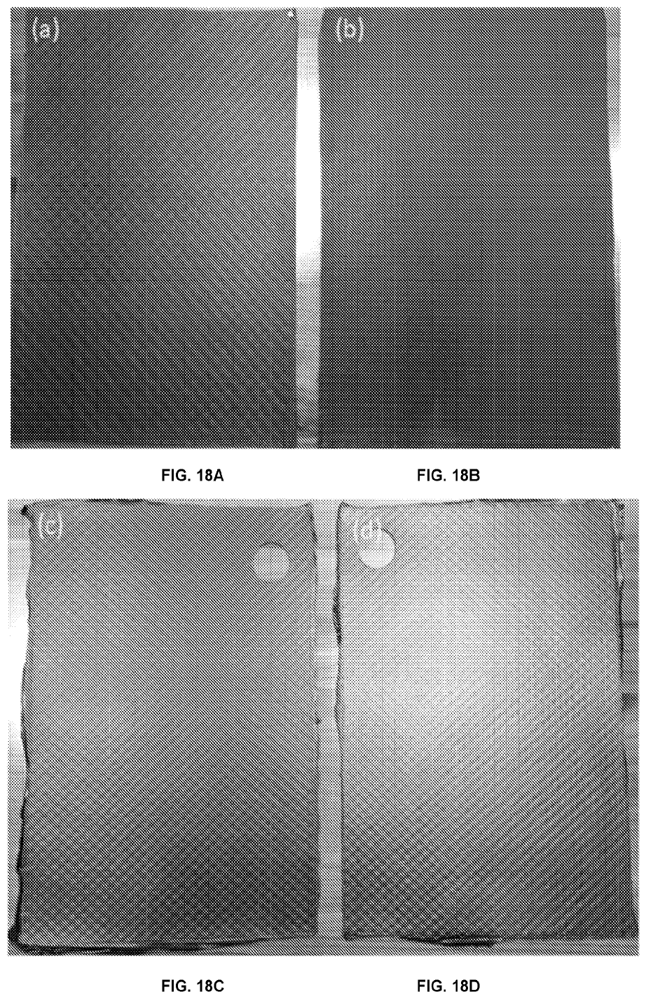

FIG. 18A-18D illustrate examples of finished wet emboss samples with the emboss pattern showing through the finish layer. FIG. 18A shows a sample wet embossed with a mold finished on the top surface. FIG. 18B shows a sample wet embossed with similar mold geometry as in FIG. 18A but with double the feature density, finished on the top surface. FIG. 18C shows a sample wet embossed with the drawer liner that is finished on the top surface. FIG. 18D shows a sample wet embossed with the drawer liner finished on the embossed surface.

Surface defects such as pits, valleys, or crinkles may show through the finish layer, visually interfering with any emboss pattern on the finish layer. Even if the surface is relatively smooth, local density differences across the surface may still show through the finish layer, which will also visually interfere with any emboss pattern on the finish layer. As such, materials should be processed to provide the desired defective areas while maintaining a suitable smoothness and visual appearance on the finished surface.

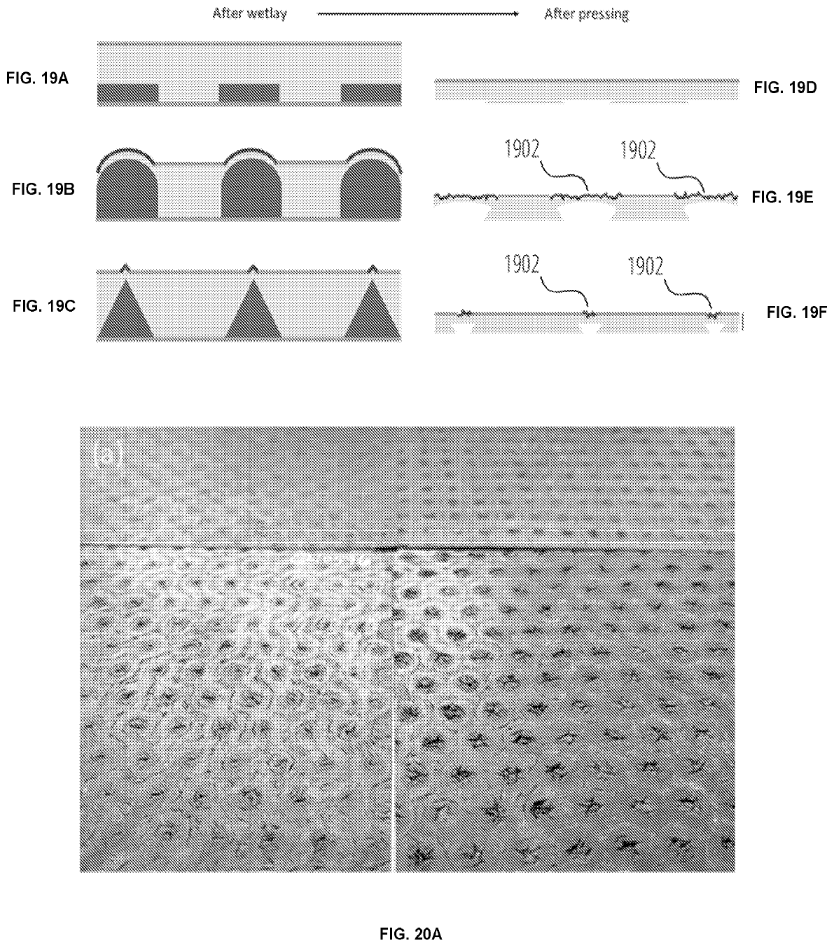

FIG. 19A-19F show a schematic illustration of how mold feature height affects surface smoothness. FIG. 19A shows a mold with short features after wetlay. FIG. 19D shows that the short features mold produces a shallow emboss but a relatively thick undisrupted top layer after pressing. FIG. 19B shows a mold with tall domed features which extend through the thickness of the web after wetlay. FIG. 19E shows that the mold with tall domed features, after pressing, produces the thin areas outlined and indicated as wrinkles 1902. The wrinkles 1902 reducing surface smoothness. FIG. 19C shows a mold with features the same height as in FIG. 19B, but with reduced volume. This increases the volume fraction of non-embossed, non-deformed material and reduces the severity of surface wrinkles 1902 as shown in FIG. 19F. As shown in FIGS. 19A-19F, increasing the volume fraction of non-embossed, non-deformed material, such as in the region along the top surface, can provide a surface that has fewer visible deformations where disruption areas are visible from the top surface.

FIG. 20A-20B illustrate examples of top surface morphology. It is hypothesized that balancing the disruption area embossed thickness with the thickness of the undisrupted top layer is important for controlling the surface smoothness. As such, the embossed deformed volume fraction should remain low enough to allow for an undisrupted top layer. FIG. 20A shows samples with top surface morphology similar to popped bubble wrap. The molds used for these samples were pyramids or pillars 3-6 mm tall. Tall mold features or a mold that occupies a high volume fraction of the web and that extend through the top surface tend to leave a very thin layer of material that collapses and wrinkles up pressing, giving a popped bubble wrap morphology. FIG. 20B shows a wet embossed sample with a smooth top surface. The mold used for this sample was 1.8 mm thick polyethylene netting with 3 mm square holes. As shown in FIG. 20B, short mold features or a mold that occupies a low volume fraction and that does not reach the top of the web leaves a substantial undisrupted layer which is more like the surface of a standard web.

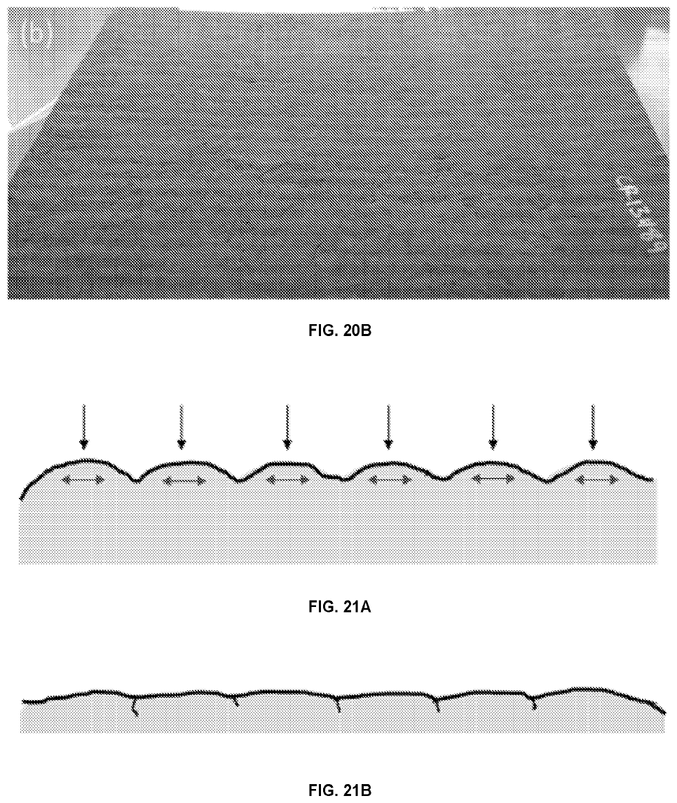

FIG. 21A-21B illustrate a schematic showing impingement of densely packed surface features upon pressing, leading to a macroscopically flat surface but with fine cracks where the compressed features meet. FIG. 21A shows the wetlay during pressing. FIG. 21B shows the wetlay after pressing. In the case where a wet embossed web has protruding hills on its surface that are also densely packed, the impingement of these hills upon pressing can give the surface a cracked appearance. Such cracks may show through after finishing or cause bridge bubbles. This effect is shown in FIG. 21B.

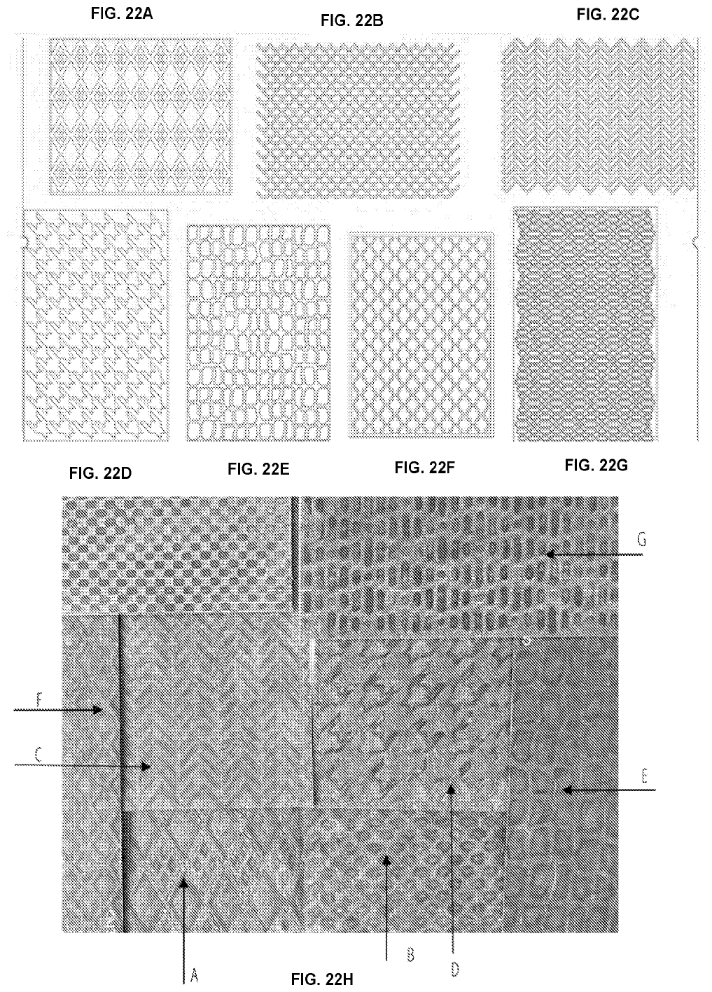

FIG. 22A-22H shows another approach to finishing wet emboss samples. This process uses wet embossing to impart an intentional aesthetic pattern to the material that will show through the finish. FIG. 22A-22G show laser cut acrylic molds with different patterns. FIG. 22A shows mold with an argyle pattern. FIG. 22B shows a mold with a fish scale pattern. FIG. 22C shows a mold with a herringbone pattern. FIG. 22D shows a mold with a houndstooth pattern. FIG. 22E shows a mold with a crocodile pattern. FIG. 22F shows a mold with a quatrefoil pattern. FIG. 22G shows a mold with a sharkshin pattern. The resulting crust materials are shown in FIG. 22H.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### Materials and Methods

The following material and methods were used.

### Mold Design and Fabrication.

CAD models for laser cut and 3D printed molds were generated using Autodesk Fusion 360. Laser cut molds were fabricated from cell cast acrylic sheets. 3D printing molds were printed in PLA or nylon 12 using either fused deposition modeling or selective laser sintering, respectively.

### Biomass Bleaching.

Prior to use, pulped biomass was bleached with hydrogen peroxide to lighten its color. Hydrogen peroxide was added to a 2.5% w/w aqueous slurry of pulped biomass to a final concentration of 2% (w/w) and left to stir for 16 hours. Afterwards, catalase was added to the slurry and mixed until the final concentration of hydrogen peroxide was less than 10 ppm. The bleached slurry was then dewatered via filter press, resuspended in water, and dewatered again via wetlay. The resulting biomass webs were dried at 60° C. for 8 hours. The final bleached and dried biomass webs were stored and used as input material for later experiments.

### Dyeing.

Bleached biomass was soaked in water along with nanofibrillated cellulose (NFC, Grade L-115-6, Engineered Fibers Technology) for 30 minutes, followed by blending using a Blendtec Pro 800 blender to create an aqueous slurry consisting of 2.2% (w/w) biomass and 0.75% NFC. Separately, a 0.46% (w/w) slurry of 10 mm 1.15 dtex lyocell fiber was first blended to disperse the fiber bundles, and then stirred with an overhead mixer at 250 RPM for 5 minutes. The biomass/NFC slurry was then added to the lyocell fiber slurry along with sodium sulfate (final concentration 1.8% (w/v) and the mixture was heated to 50° C. while stirring at 250 RPM. Upon reaching 50° C., dye (Avitera Black SE, Huntsman) was added to the mixture up to a concentration of 0.2% (w/v) and left to stir for 15 minutes. Afterwards, sodium carbonate was added (final concentration 0.5% w/v) to raise the pH and fix the dye to the surface of the biomass and fibers. After 15 more minutes of stirring, the slurry was removed from heating and dewatered via wetlay, followed by resuspension in fresh water to its original concentration. A total of 4 dewatering/resuspension cycles was performed

### Formulation.

Binder (Dur-O-Set E230, Celanese), softener (Starsoft BIS-45, StarChem), and defoamer (Entschäumer BOS, Polycoating) was blended with the dyed and washed slurry. Water was then added to the mixture to achieve the final concentrations listed in Table 1.

TABLE 1
Concentration of each slurry component
after formulation and prior to wetlay.

| Component | Final concentratio (% w/w) |
|---|---|
| Biomass, NFC, and fibers (combined) | 0.9% |
| Dur-O-Set E230 | 0.8% (wet basis) |
| Starsoft BIS-45 | 0.7% (wet basis) |
| Entschäumer BOS | 0.01% (wet basis) |

Wetlay with Wet Embossing.

Wetlay was performed using a custom batch wetlay apparatus. The apparatus consisted of a polycarbonate or polypropylene box, inside which a papermaking screen (also referred to as a forming cloth) and deckle were supported. The box was connected to a vacuum source and liquid trap and was fitted with a gauge to monitor the pressure. Before wetlay, a mold was placed on top of the forming cloth. The formulated slurry was then carefully poured into the box and over the mold and forming cloth. Dewatering was achieved by applying vacuum between −50 to −80 mbar until the moisture content of the resulting web reached steady state, approximately 90%. Afterwards, the web and mold were removed from the box, the mold was peeled off of the web, and the web was dried in a dehydrator for 10 hours at 60° C. Typical samples disclosed herein hac an average final thickness of 0.8 mm-1.0 mm while samples described herein as thin had an average final thickness of 0.3 mm-0.4 mm.

### Mechanical Testing.

Prior to all mechanical testing, samples were conditioned at 50% RH and 25° C. for 24 hours. All mechanical testing was performed on Zwick Universal Testing Machines at 50% RH and 25° C.

### Peel Resistance (T-Peel)

T-peel testing was performed according to ASTM D176 with modification. To prepare T-peel specimens, samples were cut into 0.5″×3″ strips and adhered to a similarly sized strip of ⅛″ thick neoprene rubber using cyanoacrylate glue, leaving a 0.5″ tab unglued so that the sample and the rubber could be held in the top and bottom grips of the Universal Testing Machine (from Zwick?). The rubber was scuffed using 80 grit sandpaper before use to remove bloom and increase the surface area for adhesion. The sample-rubber laminates were clamped together using spring clamps overnight to ensure good contact while the adhesive cured. Afterwards, T-peel testing was performed as specified in ASTM D1876.

### Single Edge Tear.

Single edge tear testing was performed according to ISO 3377-1.

### Tensile Testing.

Tensile tests were performed according to ISO 3376 with modification. To prepare tensile test specimens, samples were cut into 10 mm×40 mm rectangles. Tests were only considered valid if the fracture occurred between the grips.

### 3-Point Bending.

3-point bending tests were performed according to ASTM D790.

### Scanning Electron Microscopy (SEM) Imaging and Fourier Transform (FT) Analysis

Scanning electron microscopy (SEM) used a focused electron beam to assess the morphology of materials through the detection of secondary and/or backscattered electrons. The electron beam was scanned in a raster pattern to collect micrographs at scales between 1 mm and 10 nm or between 10× and 100,000× magnification. The SEM method used low vacuum (1 to 10 torr), avoiding the need for dehydrating or sputter coating biological samples.

Prior to imaging, samples were cross sectioned using a clean teflon-coated razor and mounted on the SEM stage using carbon tape. SEM was performed with a Hitachi TM3030 Plus environmental scanning electron microscope at an accelerating voltage of 15 kV using mixed (backscattered and secondary electron) imaging.

### Example 1: SEM Micrographs of Composite Mycelium Materials

Composite mycelium material that had not been wet embossed had a relatively uniform structure with fibers orientated predominantly in the x-y plane as shown in FIG. 2A. Fibers lay on top of each other and parallel to the forming cloth during the wetlay process. Only a few fibers appear to bridge the peel gap as a result of a manual peel as shown in FIG. 7B. Crack and peel propagation tend to follow the path of least resistance. Mycelium fibers and additive fibers of composite mycelium material samples tend to lie in the x/y plane.

Mycelium material that had been wet embossed showed fibers with varying degrees of undulation or folding in the deformation area as shown in FIG. 2B. Deformation areas were formed during the wetlay process as the fibers lay over the features of the mold and then were deformed during pressing. FIGS. 7C and 7D show SEM micrographs of a composite mycelium material sample produced using wet embossing techniques subjected to a manual peel. Delamination caused by the peel is shown as dashed arrows in FIG. 7C as the folded over deformation area was pulled out of the bottom, and how the peel propagation path avoids the dense area in FIG. 7D.

Typically the deformation area does not extend to the top surface as the top surface is preferably generally smooth and uniform. In cases where the top surface has deep pits or valleys, applied finish layers may not evenly coat the surface, resulting in undesirable bubbles. FIGS. 17A and 17B show a mycelium material with a non-uniform surface and a finish layer with small bubbles.

### Example 2: Tensile Properties of Needled Composite Mycelium Material Samples

By incorporating a step of wet embossing, the resulting average peel force (N/cm) of mycelium samples that have been wet embossed was significantly higher than that of mycelium samples that were not wet embossed.

Representative force-travel plots from T-peel tests performed on standard and wet embossed samples are shown in FIG. 8A-D. Each of the wet embossed samples show distinct periodic peaks in force, corresponding to individual embossed features being pulled apart. Notably the standard wetlay sample shown in FIG. 8A lacks these periodic peaks, which demonstrates how wet embossing can measurably increase the peel resistance of a sample.

In addition, the peaks in the wet emboss sample embodiments of the invention described where the mold was laid on top of the slurry prior to dewatering were less distinct than the wet emboss sample embodiments of the invention where the slurry was poured on top of the mold as shown in FIG. 8C. These results indicate that the features in the wet emboss sample embodiments of the invention, where the mold is laid on top of the slurry were similarly less distinct.

Samples were prepared containing 21% and 26% lyocell fibers were produced as described above. Both samples were wet embossed over molds. FIG. 8B indicates samples having 21% lyocell produced more pronounced peaks and a higher peel force (N/cm) than similar samples containing 26% lyocell. The average final thickness of the samples was 0.8 mm-0.1 mm.

FIGS. 9C and D show the average peel force (N/cm) of composite mycelium materials prepared using a mold consisting of closely spaced parallel slats running in the x-direction of the web. After pressing, the emboss features fold over and form a series of parallel pleats.

When testing parallel along a pleat, the T-peel force is sustained and consistently high (average 8.1 N/cm) as shown in FIG. 9C. When testing perpendicular to the pleats, the force spikes when encountering a pleat and drops significantly when traveling between them, leading to a lower average force (3.6 N/cm) as shown in FIG. 9D.

### Example 3: Wet Embossing of the Composite Mycelium Material with Different Mold Types

Numerous different types of embossing molds were investigated. The features on the embossing molds were different sizes and shapes, and were spatially positioned at different locations. Experiments were also performed with the composite mycelium material being wetlaid on top of the molds, other experiments the molds were pressed onto the top wetlaid composite mycelium material. In other experiments, the molds projected through the wetlaid composite mycelium material.

FIG. 11 shows a heatmap of feature forming ability as a function of (straight-walled) hole diameter and depth. For the baseline composition, features were successfully formed in the 6-9 mm ranges. Images are provided for visual reference only and are not drawn to scale.

FIGS. 12A and C show molds with straight-walled features. Depending on the aspect ratio of the holes, the slurry may not be able to effectively fill them. FIGS. 12B and D show molds with features of the same spacing, base area, and height as in FIGS. 12A and C, but with slanted walls. This increases the effective hole diameter and volume of the hole, allowing the slurry to fill in the entire hole more easily.

FIG. 13A shows features spaced apart that give rise to a discontinuous curvature as shown in FIG. 13C. FIG. 13B shows features densely packed that give rise to a continuous curvature as shown in FIG. 13D.

FIG. 19A-F show a schematic illustration of how mold feature height affects surface smoothness. FIG. 19A shows a mold with short features after wetlay. FIG. 19D shows that the short features mold produced a shallow emboss but a relatively thick undisrupted top layer after pressing. FIG. 19B shows a mold with tall domed features which extend through the thickness of the web after wetlay. FIG. 19E shows that the mold with tall domed features, after pressing, produced the thin areas outlined and indicated as wrinkles 1902. The wrinkles 1902 reducing surface smoothness. FIG. 19C shows a mold with features the same height as in FIG. 19B, but with reduced volume. This increased the volume fraction of non-embossed, non-deformed material, and reduced the severity of surface wrinkling as shown in FIG. 19F.

### Example 4: Mold Design Parameters and their Effect on Processing and Properties

### Mold Material and Fabrication Methods

In addition to choosing mold geometry for the desired mechanical and aesthetic properties, molds should be designed to be easy to unmold from a wet web. This is facilitated by: a smooth mold surface and mold material surface chemistry which minimize adhesion to the wet web, and mold flexibility and features that facilitate removal from the web.

The different mold materials and fabrication methods tested include laser cut acrylic, 3D-printed using fused deposition modeling and polylactic acid (PLA), 3D-printed using selective laser sintering and nylon, and off the shelf materials such as drawer liners, rug underlays, meshes and netting,

### Unmolding Techniques

At research scale, unmolding after the wetlay embodiment of the invention described in FIG. 3A-B was done in one of two ways:

1. Manual unmolding: The web and mold were removed from the wetlay box, and inverted to remove the forming cloth. The mold was then carefully peeled off the web. This technique was generally used when the mold was flexible and the features release easily from the web.

2. Vacuum-assisted unmolding: The web and mold were removed from the wetlay box, inverted, and carefully placed back into the wetlay box. Vacuum was applied to hold the wet web in place while the mold (now exposed) was removed. This technique was generally used when the mold was inflexible and/or the features did not release easily from the web.

Thus far, no significant difference in aesthetics or mechanical properties have been observed between samples made with the two unmolding techniques. Web cohesiveness facilitates unmolding and reducing the cohesiveness of the wet web makes unmolding more difficult. Dewatering to steady state, approximately 90% water content or below and adding long fibers such as NFC or lyocell improve web cohesiveness and facilitate unmolding. At high water content or in the absence of long fibers, the web is much more likely to tear or stretch during unmolding, and/or delaminate, with parts of the web sticking to the mold as it is removed.

### Example 5: The Effect of Mold Geometry on Mechanical Properties

The effect of mold geometry on mechanical properties can be observed from the results of experiments which used 3D printed pillar arrays as molds. These molds spanned a range of feature spacings, heights, and sizes from 2-8 mm. In addition, feature shape (ex. pillars vs cones vs pyramids), lattice pattern (square vs hexagonal arrangements) and the extent of feature draft/chamfer were varied. Overall, most mechanical properties were not strongly affected by these parameters. Details of the molds used are described in Table 3A. The summary statistics for all of the samples are shown in Table 3B.

TABLE 3A
Summary of molds used to assess effect of mold geometry on sample mechanical
properties. The feature base major dimension refers to the diameter for pillar
or cone features, and the side length for square or pyramid features. The
number of samples made with each mold refers to the number of wetlays where
the mold was used; the resulting material was subdivided into individual
test specimens for various mechanical tests as shown in Table 3B.

| | | | | | Feature | Number |
|---|---|---|---|---|---|---|
| | | Feature | Feature | Feature | base major | of samples |
| Mold | Feature | lattice | spacing | height | dimension | made with |
| ID | type | pattern | (mm) | (mm) | (mm) | mold |
| 007-1 | Domed | Hexagonal | 4 | 3.5 | 5 | 1 |
| | pillar | | | | | |
| 007-2 | Cone | Hexagonal | 4 | 5.5 | 5 | 1 |
| 007-3 | Domed | Hexagonal | 4 | 5.5 | 5 | 1 |
| | pillar | | | | | |
| 007-4 | Domed | Hexagonal | 2 | 3.5 | 5 | 1 |
| | pillar | | | | | |
| 007-5 | Square | Square | 4 | 4 | 3.5 | 1 |
| | pillar | | | | | |
| 007-6 | Square | Square, rotated | 4 | 4 | 3.5 | 1 |
| | pillar | 45 deg | | | | |
| 007-7 | Square | Square, rotated | 4 | 5.5 | 4 | 1 |
| | pyramid | 45 deg | | | | |
| 007-7-1 | Square | Square, rotated | 4 | 3.5 | 4 | 2 |
| | pillar | 45 deg | | | | |
| 007-7-2 | Square | Square, rotated | 4 | 3.5 | 4 | 2 |
| | pyramid | 45 deg | | | | |
| 015-1 | Cone | Hexagonal | 2 | 1 | 2 | 1 |
| 015-2 | Cone | Hexagonal | 2 | 1 | 2 | 1 |
| 015-3 | Cone | Hexagonal | 2 | 1 | 8 | 1 |
| 015-4 | Cone | Hexagonal | 2 | 1 | 8 | 1 |
| 015-5 | Cone | Hexagonal | 8 | 1 | 2 | 1 |
| 015-6 | Cone | Hexagonal | 8 | 1 | 2 | 1 |
| 015-7 | Cone | Hexagonal | 8 | 1 | 8 | 1 |
| 015-8 | Cone | Hexagonal | 8 | 1 | 8 | 1 |

TABLE 3B
Summary statistics for all specimens tested which used 3D
printed pillar array molds. N refers to the number of test
specimens. Standard wetlay data is shown for comparison.

| | | | | | Baseline |
|---|---|---|---|---|---|
| | Test wet embossed | | | | (standard |
| | 1st | | 3rd | | wetlay) |
| Property | quartile | Median | quartile | n | Mean ± SD |
| Single edge tear average | 7 | 8.6 | 10 | 37 | 6.5 ± 1 |
| force (N) | | | | | |
| Dry tensile strength (MPa) | 7.4 | 10.2 | 11.8 | 100 | 10 ± 0.8 |
| Dry tensile elongation at | 37 | 41 | 46 | 100 | 27 ± 5 |
| break (%) | | | | | |
| Dry tensile initial modulus | 17 | 23 | 30 | 100 | 51 ± 7 |
| (MPa) | | | | | |
| Average T-peel force, top | 3.2 | 3.8 | 4.3 | 64 | 2.6 ± 0.4 |
| side (N/cm) | | | | | |
| Average T-peel force, | 4.4 | 4.9 | 5.5 | 99 | |
| embossed side (N/cm) | | | | | |
| Dry bally flex cycles | — | 10000 | — | 6 | 10000 ± 0 |
| completed before failure | | | | | |
| (examination interval 10k | | | | | |
| cycles) | | | | | |

### Example 6: Effect of Input Mass on Wet Emboss and Mechanical Properties

Samples were prepared with reduced input masses (42% of baseline) to create thin material with an average final thickness of 0.3 mm-0.4 mm. For this experiment, a rug underlay (same pattern as drawer liner but only 2 mm thick) was used as a mold. The mean and standard deviation of the mechanical properties measured are shown in Table 4. Notably, the wet embossed samples have significantly higher dry tensile elongation at break than the standard sample.

TABLE 4
Mechanical properties (mean +/− standard deviation)
for thin samples with and without wet emboss.

| | | Dry tensile | Dry tensile | Average single | |
|---|---|---|---|---|---|
| | Dry tensile | elongation at | initial modulus | edge tear force | Average T-peel |
| Sample | strength (MPa) | break (%) | (MPa) | (N) | force (N/cm) |
| Non-wet | 13.7 +/− 0.3 | 23 +/− 2 | 25 +/− 2 | 3.2 +/− 0.2 | N/A |
| embossed thin | | | | | |
| material | | | | | |
| Wet embossed | 10 +/− 1 | 36 +/− 4 | 26 +/− 5 | 3.5 +/− 0.9 | 3 +/− 0.4 |
| thin material | | | | | |
| #1 | | | | | |
| Wet embossed | 10 +/− 1 | 38 +/− 4 | 19 +/− 4 | 3.9 +/− 0.2 | 2.6 +/− 0.6 |
| thin material | | | | | |
| #2 | | | | | |

In thin samples, the maximum amplitude of an undulation and deformation area volume was limited by the sample thickness. This reduces the degree to which an embossed feature can be further deformed upon pressing, as shown in FIGS. 16B-C. This can reduce peel resistance compared to a thicker sample.

Aspects:

Aspect 1. A mycelium material comprising mycelium fibers, the mycelium material being planar and having a first surface, a second surface opposite the first surface, and a thickness, the first surface comprising one or more deformation areas spaced apart from each other, wherein the one or more deformation areas do not extend from the first surface to the second surface.

Aspect 2. The mycelium material of aspect 1, wherein the one or more deformation areas have a higher density than surrounding areas.

Aspect 3. The mycelium material of aspect 1 or 2, wherein a majority of the mycelium fibers are orientated in an x-y plane and the one or more deformation areas comprise one or more disrupted fibers that are oriented in a z-direction.

Aspect 4. The mycelium material of any one of the preceding aspects, wherein the one or more deformation areas are discrete deformation areas.

Aspect 5. They mycelium material of any one of the preceding aspects, wherein the one or more deformation areas are continuous deformation areas.

Aspect 6. The mycelium material of any one of the preceding aspects, wherein the mycelium material is a composite mycelium material that comprises a bonding agent and an additive.

Aspect 7. The mycelium material of aspect 6, wherein the bonding agent comprises an adhesive, a resin, a crosslinking agent, and/or a matrix.

Aspect 8. The mycelium material of aspect 6, wherein the bonding agent is selected from the group consisting of a vinyl acetate-ethylene (VAE) copolymer, a vinyl acetate-acrylic copolymer, a polyamide-epichlorohydrin resin (PAE), a copolymer, transglutaminase, citric acid, genipin, alginate, gum arabic, latex, a natural adhesive, and a synthetic adhesive.

Aspect 9. The mycelium material of any one of the preceding aspects, wherein the mycelium material is a composite mycelium material that comprises a reinforcing material.

Aspect 10. The mycelium material of any one of the preceding aspects, wherein the mycelium material further comprises a plasticizer.

Aspect 11. The mycelium material of any one of the preceding aspects, further comprising a lyocell fiber or an abaca fiber.

Aspect 12. The mycelium material of aspect 3, wherein the one or more disrupted fibers have a length of 0.1 mm to 5 mm.

Aspect 13. A mycelium material comprising mycelium fibers, the mycelium material being planar and having a first surface, a second surface opposite the first surface, and a thickness, the mycelium material having a first region having a first density, and a second region having a deformation area extending from the first surface toward the second surface, wherein the second region has a second density that is higher than the first density.

Aspect 14. The mycelium material of aspect 13, wherein the mycelium material has a plurality of deformation areas and a deformed volume fraction of less than about 50% of the mycelium material, less than about 40% of the mycelium material, less than about 30% of the mycelium material, or less than about 20% of the mycelium material.

Aspect 15. The mycelium material of aspect 13 or 14, wherein the deformation area is a discrete deformation area or a continuous deformation area.

Aspect 16. The mycelium material of any one of aspects 13 to 15, wherein the mycelium material is a composite mycelium material comprising a bonding agent and an additive.

Aspect 17. A method of producing a mycelium material, the method comprising:
providing mycelium fibers;
adding a solution to the mycelium fibers to produce a mycelium composition;
wet embossing the mycelium composition such that one or more deformation areas are formed; and
drying the mycelium composition; thus producing the mycelium material.

Aspect 18. The method of aspect 17, wherein a bonding agent is added to the mycelium composition before wet embossing.

Aspect 19. The method of aspect 17, wherein the mycelium material is planar and has a first surface, a second surface opposite the first surface, and a thickness, and the one or more deformation areas are discrete deformation areas that are spaced apart from each other and that do not extend from the first surface to the second surface.

Aspect 20. The method of aspect 17, wherein the mycelium material is planar and has a first surface, a second surface opposite the first surface, and a thickness, and the one or more deformation areas are continuous deformation areas that are interconnected with each other and that do not extend from the first surface to the second surface.

1. A mycelium material comprising mycelium fibers, the mycelium material being planar and having a first surface, a second surface opposite the first surface, and a thickness, the first surface comprising one or more deformation areas spaced apart from each other, wherein the one or more deformation areas do not extend from the first surface to the second surface.

2. The mycelium material of claim 1, wherein the one or more deformation areas have a higher density than surrounding areas.

3. The mycelium material of claim 1, wherein a majority of the mycelium fibers are orientated in an x-y plane and the one or more deformation areas comprise one or more disrupted fibers that are oriented in a z-direction.

4. The mycelium material of claim 1, wherein the one or more deformation areas are discrete deformation areas.

5. They mycelium material of claim 1, wherein the one or more deformation areas are continuous deformation areas.

6. The mycelium material of claim 1, wherein the mycelium material is a composite mycelium material that comprises a bonding agent and an additive.

7. The mycelium material of claim 6, wherein the bonding agent comprises an adhesive, a resin, a crosslinking agent, and/or a matrix.

8. The mycelium material of claim 6, wherein the bonding agent is selected from the group consisting of a vinyl acetate-ethylene (VAE) copolymer, a vinyl acetate-acrylic copolymer, a polyamide-epichlorohydrin resin (PAE), a copolymer, transglutaminase, citric acid, genipin, alginate, gum arabic, latex, a natural adhesive, and a synthetic adhesive.

9. The mycelium material of claim 1, wherein the mycelium material is a composite mycelium material that comprises a reinforcing material.

10. The mycelium material of claim 1, wherein the mycelium material further comprises a plasticizer.

11. The mycelium material of claim 6, wherein the additive comprises a lyocell fiber or an abaca fiber.

12. The mycelium material of claim 3, wherein the one or more disrupted fibers have a length of 0.1 mm to 5 mm.

13. A mycelium material comprising mycelium fibers, the mycelium material being planar and having a first surface, a second surface opposite the first surface, and a thickness, the mycelium material having a first region having a first density, and a second region having a deformation area extending from the first surface toward the second surface, wherein the second region has a second density that is higher than the first density.

14. The mycelium material of claim 13, wherein the mycelium material has a plurality of deformation areas and a deformed volume fraction of less than about 50% of the mycelium material, less than about 40% of the mycelium material, less than about 30% of the mycelium material, or less than about 20% of the mycelium material.

15. The mycelium material of claim 13, wherein the deformation area is a discrete deformation area or a continuous deformation area.

16. The mycelium material of claim 13, wherein the mycelium material is a composite mycelium material comprising a bonding agent and an additive.

17. A method of producing a mycelium material, the method comprising:
providing mycelium fibers;
adding a solution to the mycelium fibers to produce a mycelium composition;
wet embossing the mycelium composition such that one or more deformation areas are formed; and
drying the mycelium composition; thus producing the mycelium material.

18. The method of claim 17, wherein a bonding agent is added to the mycelium composition before wet embossing.

19. The method of claim 17, wherein the mycelium material is planar and has a first surface, a second surface opposite the first surface, and a thickness, and the one or more deformation areas are discrete deformation areas that are spaced apart from each other and that do not extend from the first surface to the second surface.

20. The method of claim 17, wherein the mycelium material is planar and has a first surface, a second surface opposite the first surface, and a thickness, and the one or more deformation areas are continuous deformation areas that are interconnected with each other and that do not extend from the first surface to the second surface.
